(19) United States
(12) Patent Application Publication
(10) Pub. No.: US 2022/0211612 A1
(43) Pub. Date: Jul. 7, 2022

(54) GEL BASE COMPOSITION FOR COMPOUNDING INTO A MUCOADHESIVE DELIVERY SYSTEM

(71) Applicant: MEDISCA PHARMACEUTIQUE Inc., St-Laurent (CA)

(72) Inventors: Panagiota Danopoulos, Montreal (CA); Rodrigo Lupatini, Plattsburgh, NY (US)

(21) Appl. No.: 17/703,427

(22) Filed: Mar. 24, 2022

Related U.S. Application Data
(63) Continuation of application No. PCT/CA2020/051278, filed on Sep. 24, 2020.
(60) Provisional application No. 62/904,968, filed on Sep. 24, 2019.

(51) Int. Cl. A61K 9/00; A61K 45/06; A61K 47/36; A61K 47/42; A61K 47/10

(52) U.S. Cl. CPC A61K 9/006; A61K 45/06; A61K 47/10; A61K 47/42; A61K 47/36

(57) ABSTRACT

The present invention relates to mucoadhesive concentrated base compositions having high viscosity of at least 50,000 cPs, which can be processed upon dilution into a gel dosage form and upon drying into a strip dosage form for use as a vehicle for delivery of active ingredients to mucocutaneous surfaces, such as the oral, rectal, nasal or vaginal cavities.

### CROSS-REFERENCE TO RELATED APPLICATION

The present application claims the benefit of U.S. provisional patent application Ser. No. 62/904,968 filed on Sep. 24, 2019. The contents of the above-referenced document are incorporated herein by reference in their entirety.

### TECHNICAL FIELD

This application generally relates to the field of mucoadhesive compounding formulations for administration of active ingredients.

### BACKGROUND

An important requirement of drug delivery technology is the formulation of a delivery system that is capable of achieving a desirable release profile for the ever-increasing number of active ingredients (e.g., pharmaceutical, cosmetic, cannabinoids, nutraceutical, etc.) with limited to poor water solubility. Bioadhesive drug delivery systems have been proposed as an improved administration route whereby one takes advantage of natural or synthetic materials to adhere to biological membranes, resulting in intimate contact of the material with the tissue for a more or less prolonged period of time.

A particular case of bioadhesive drug delivery system includes mucoadhesive drug delivery systems, where the tissue is a mucous membrane such as those found, for instance, in the oral, vaginal, rectal and nasal cavities. Bioadhesion can be defined as the state in which two materials, at least one of which is biological in nature, are maintained together for a prolonged time period by means of interfacial forces. Accordingly, the use of bioadhesive drug delivery systems may result in an increase in drug bioavailability.

Mucoadhesive drug delivery systems in the past have been formulated as powders, compacts, gels, sprays, or semisolids. For example, compacts have been used for drug delivery to the oral cavity, and powders and nanoparticles have been used to facilitate drug administration to the nasal mucosa. Recently oral film strips were developed for tongue or buccal cavity. Buccal films have been suggested as a means of offering greater flexibility and comfort than adhesive tablets. An additional advantage for these dosage forms, when compared to tablets, capsules and other dosage forms that must be swallowed, is that some patient populations have difficulty swallowing, such as children and the elderly.

Several gel or film dosage forms have, thus, been extensively developed for the treatment of oral, vaginal, rectal and nasal cavity diseases.

There are, however, many major difficulties and challenges associated with the manufacture of mucoadhesive film dosage forms ranging from brittleness, tackiness, the hygroscopic nature and potential lack of homogeneity within the dosage form, and the like. Ideal physical characteristics of the film include dosage uniformity throughout, adequate flexibility and tensile strength to facilitate processing, handling, and packaging of the film in a consumer-friendly form. Attaining ideal conditions for one characteristic usually comes at the expense of other, often equally important, properties, resulting in a necessary compromise in various properties to achieve a working film dosage form. Therefore, the main challenges and obstacles encountered when using film technology as a delivery vehicle are due to the very properties upon which oral film technology is based. For example, challenges are encountered when attempting to provide an oral dosage as a film exhibiting a high content of liquid ingredients (0-35% wt/wt), and high drug loading in a matrix which is formulated as a very thin (under 80 micron) and continuous, yet flexible film layer.

While gels have the advantage of easy dispersion throughout the mucosal surface and the application of mucoadhesive gels provide an extended retention time in the mucosal cavity, adequate drug penetration, as well as high efficacy and patient compliance, there are also many major difficulties and challenges associated with the manufacture of gel dosage forms. For example, because gels are networks of polymer chains that are sometimes found as colloidal gels in which water is the dispersion medium, this limits drug compatibility to the realm of hydrophilic drugs.

Despite the above discussed need for drug delivery systems, mucoadhesive drug delivery technologies with wide drug compatibility and which address at least some of the problems discussed above remains elusive.

### SUMMARY

This Summary is provided to introduce a selection of concepts in a simplified form that are further described below in the Detailed Description. This Summary is not intended to identify key aspects or essential aspects of the claimed subject matter.

As embodied and broadly described herein, the present disclosure relates in one broad aspect to compositions having high viscosity of at least 50,000 cPs, which can be processed upon dilution into a gel dosage form and upon drying into a strip dosage form. Such dosage forms can be used as a vehicle for delivery of active ingredients to mucocutaneous surfaces, such as the oral, rectal, nasal or vaginal cavities.

As embodied and broadly described herein, the present disclosure relates in one broad aspect to a composition for use in formulating a mucoadhesive delivery dosage form, the composition having a viscosity of at least 50,000 cPs, wherein upon spreading the composition as a layer on a substrate, the composition optionally having been diluted with a diluting agent, and drying the composition, the composition being compoundable into a mucoadhesive film strip dosage form, and wherein upon dilution with a diluting agent, the composition being compoundable into a mucoadhesive gel dosage form.

As embodied and broadly described herein, the present disclosure also relates in one broad aspect to a composition for use in formulating a mucoadhesive delivery dosage form, the composition being compatible with a hydrophilic and a hydrophobic active ingredient, wherein upon drying the composition being compoundable into a film strip dosage form and, upon dilution, into a gel dosage form.

As embodied and broadly described herein, the present disclosure also relates in one broad aspect to a composition for use in formulating a mucoadhesive delivery dosage form, the composition having a viscosity of at least 50,000 cPs, wherein upon dilution the composition being in a gel dosage form, and upon spreading the composition as a layer on a substrate, the composition optionally having been diluted with a diluting agent, and drying the composition, the composition being in a film strip dosage form.

In one non-limiting embodiment, the herein described dilution can be performed with a suitable diluting agent. For example, a carrier, excipient, or diluent, in the form of a liquid, a cream or gel.

As embodied and broadly described herein, the present disclosure also relates in one broad aspect to a gel base composition for use in formulating a mucoadhesive delivery dosage form having a target viscosity, the composition comprising a mucoadhesive polymer dispersed in an aqueous solvent and having an initial viscosity higher than the dosage form target viscosity.

As embodied and broadly described herein, the present disclosure also relates in one broad aspect to a composition for use in formulating a mucoadhesive delivery dosage form having a target viscosity, the composition comprising a mucoadhesive polymer, a plasticizer, a pharmaceutically acceptable polyhydric alcohol and an emulsifier, the composition having an initial viscosity higher than the dosage form viscosity.

As embodied and broadly described herein, the present disclosure also relates in one broad aspect to a composition for use in formulating a mucoadhesive delivery dosage form, the composition comprising a mucoadhesive polymer and being compoundable into a hydrogel dosage form having an average maximum compressive force ≤−3.0 g and an average maximum adhesive force ≥1.8 g.

As embodied and broadly described herein, the present disclosure also relates in one broad aspect to a composition for use in formulating a mucoadhesive delivery dosage form, the composition comprising a mucoadhesive polymer and being compoundable into a strip film dosage form characterized with an average load at break ≥1000 g.

As embodied and broadly described herein, the present disclosure also relates in one broad aspect to a composition for use in formulating a mucoadhesive delivery dosage form, the composition comprising a mucoadhesive polymer and being compoundable into a strip film dosage form characterized with an average stress at break of ≥10.0×10⁷ Dynes/cm².

As embodied and broadly described herein, the present disclosure also relates in one broad aspect to a kit comprising (i) a composition for use in formulating a mucoadhesive delivery dosage form; (ii) instructions for compounding the composition into a film strip dosage form; and (iii) instructions for compounding the composition into a gel dosage form.

In one non-limiting embodiment, whether one elects to obtain the film strip dosage form or the gel dosage form can be based on the desired compounding application, which affords an advantageous dosage form flexibility.

In one embodiment, the following features, alone or in any possible combinations, may characterize the herein described composition:
a target viscosity of at least 80,000 cPs, or at least 100,000 cPs, or at least 200,000 cPs, or at least 300,000 cPs, or at least 500,000 cPs;
exposing the composition to 40° C. for 5 minutes decreases the initial viscosity to the dosage form viscosity;
the dosage form viscosity is at least 20% lower than the composition initial viscosity;
a mucoadhesive polymer, a plasticizer, a pharmaceutically acceptable polyhydric alcohol and an emulsifier;
the gel dosage form having a viscosity which is reduced by at least 20% compared to the viscosity of the composition;
the gel dosage form having a target viscosity of less than 40,000 cPs;
the mucoadhesive polymer includes a natural, semisynthetic or synthetic polymer;
the mucoadhesive polymer includes amylopectin, zein, modified zein, casein, gelatin, serum albumin, collagen, chitosan, pyrrolidones, dextrins, cellulose, dextrans, tamarind seed polysaccharide, gellan, carrageenan gum, xanthan gum, arabic gum, hyaluronic acid, polyhyaluronic acid, alginic acid, locust bean gum, pullulan, poloxamers, maltodextrins, Eudragit, guar gum, tragacanth gum, modified cellulose gum, or any combinations thereof;
the mucoadhesive polymer includes carrageenan gum, xanthan gum, locust bean gum, and pullulan;
the plasticizer includes glycerin, alkylene glycols, polyalkylene glycols, glycerol, triacetin, deacetylated monoglyceride, diethyl salate, triethyl citrate, dibutyl sebacate, polyethylene glycols, propylene glycol, or any combinations thereof;
comprising an active ingredient;
the active ingredient includes an active pharmaceutical ingredient, a nutraceutical compound, a cannabinoid, a cosmetic compound, or a combination thereof,
the film strip dosage form having a water activity of <0.6;
the pharmaceutically acceptable polyhydric alcohol includes mannitol, glucose, sucrose, dextrose, sorbitol, xylitol, maltitol, erythritol, or any combinations thereof;
the emulsifier includes a poloxamer, benzalkonium chloride, polysorbate, sodium lauryl sulfate, or any combinations thereof,
the emulsifier can be polysorbate 80;
includes a mucoadhesive polymer up to 25 wt. %, a plasticizer from 1 to 8 wt. %, a pharmaceutically acceptable polyhydric alcohol from 1 to 8 wt. % and an emulsifier from 0.2 to 1 wt. %;
the dosage form is a gel dosage form or is a film dosage form.

As embodied and broadly described herein, the present disclosure also relates in one broad aspect to a method for preparing a mucoadhesive delivery dosage form, the method comprising: providing a gel base composition having an initial viscosity; diluting a first amount of said composition with a diluting agent to obtain a gel dosage form; and drying a second amount of said composition, the second amount optionally having been diluted with a diluting agent, which is spread as a layer on a substrate to obtain a film strip dosage form, wherein each of the first and second amounts of the composition includes an active ingredient.

As embodied and broadly described herein, the present disclosure also relates in one broad aspect to a method for preparing a mucoadhesive delivery dosage form, the method comprising selecting a dosage form from a gel dosage form and a film strip dosage form, and based on the selection: dispersing an active ingredient in a base composition, the base composition having a viscosity of at least 50,000 cPs, and incorporating therein a diluting agent and an active ingredient to obtain the gel dosage form, or dispersing an active ingredient in the base composition, the base composition optionally being diluted with a diluting agent, spreading same as a layer on a substrate and drying same to obtain the film strip dosage form.

As embodied and broadly described herein, the present disclosure also relates in one broad aspect to a method for preparing a mucoadhesive delivery dosage form, the method comprising dispersing an active ingredient in a base gel composition having an initial viscosity of at least 50,000 cPs to obtain a mixture; diluting a first amount of said mixture with a diluting agent to obtain a gel dosage form having a target viscosity; and drying a second amount of said mixture, the second amount optionally having been diluted with a diluting agent, which is spread as a layer on a substrate to obtain a film strip dosage form.

As embodied and broadly described herein, the present disclosure also relates in one broad aspect to a method for preparing a mucoadhesive delivery dosage form, the method comprising providing a base gel composition having an initial viscosity of at least 50,000 cPs, optionally diluting the base gel composition with a diluting agent, dispersing an active ingredient in the base gel composition to obtain a mixture; spreading a layer of the mixture on a substrate, and drying the layer to obtain a film strip dosage form having a water activity of ≤0.6.

As embodied and broadly described herein, the present disclosure also relates in one broad aspect to a method for preparing a mucoadhesive gel dosage form, the method comprising providing a base gel composition having an initial viscosity of at least 50,000 cPs; diluting the base gel composition with a diluting agent to a viscosity of less than 40,000 cPs, wherein an active ingredient is incorporated into the base gel composition prior to, during or after diluting the base gel composition.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

### BRIEF DESCRIPTION OF DRAWINGS

A detailed description of specific exemplary embodiments is provided herein below with reference to the accompanying drawings in which:

FIG. 1 is an illustrative graph showing comparative results from a shear rate sweep experiment with a concentrated base gel in accordance with an embodiment of the present disclosure (test) and a commercially available base gel (comparative). The curves show viscosity η (Pa·s) on the y axis and shear rate γ (1/s) at 37° C. on the x axis;

FIG. 2 is an illustrative graph showing comparative results from a shear rate sweep experiment with a concentrated base gel in accordance with an embodiment of the present disclosure (test) and a commercially available base gel (comparative). The curves show normal stress N (Pa) on the y axis and shear rate γ (1/s) at 37° C. on the x axis;

FIG. 3 is an illustrative graph showing comparative results from a shear rate sweep experiment with a concentrated base gel in accordance with an embodiment of the present disclosure (test) and a commercially available base gel (comparative). The curves show complex modulus G* (Pa) on the y axis and oscillation stress (Pa) at 25° C. on the x axis;

FIG. 4 is an illustrative graph showing comparative results from a shear rate sweep experiment with a concentrated base gel in accordance with an embodiment of the present disclosure (test) and a commercially available base gel (comparative). The curves show phase angle δ (°) on the y axis and oscillation stress (Pa) at 25° C. on the x axis;

FIG. 5 is an illustrative graph showing comparative results from an oscillation frequency sweep experiment with a concentrated base gel in accordance with an embodiment of the present disclosure (test) and a commercially available base gel (comparative). The curves show storage modulus (Pa) (filled shapes) and loss modulus (Pa) (empty shapes) on the y axis and angular frequency (rad/s) at 25° C. on the x axis;

FIG. 6 is an illustrative graph showing comparative results from an oscillation frequency sweep experiment with a concentrated base gel in accordance with an embodiment of the present disclosure (test) and a commercially available base gel (comparative). The curves show tan (delta) on the y axis and angular frequency (rad/s) at 25° C. on the x axis;

FIG. 7 is an illustrative graph showing comparative results from a tribological analysis experiment with a concentrated base gel in accordance with an embodiment of the present disclosure (test) and a commercially available base gel (comparative). The curves show coefficient of friction (μ) on the y axis and sliding speed (μm/s) at 37° C. on the x axis;

FIG. 8 is an illustrative graph showing results from a rheological synergism experiment with a commercially available base gel in presence or absence of mucin. The curves show viscosity (Pa·s) on the y axis and shear rate γ (1/s) at 37° C. on the x axis;

FIG. 9 is an illustrative graph showing results from the same rheological synergism experiment as in FIG. 8 but using a concentrated base gel in accordance with an embodiment of the present disclosure in presence or absence of mucin. The curves show viscosity (Pa·s) on the y axis and shear rate γ (1/s) at 37° C. on the x axis;

FIG. 10 is an illustrative histogram showing the mean from two analyses with a concentrated base gel in accordance with an embodiment of the present disclosure (test) and a commercially available base gel (comparative);

FIG. 11 is a block diagram illustrating a process for compounding a concentrated base gel in accordance with an embodiment of the present disclosure into a film strip or gel dosage form;

FIG. 12 is a picture of a concentrated gel base being prepared to obtain a film dosage form in accordance with an embodiment of the present disclosure;

FIG. 13 is a picture of a concentrated gel base of FIG. 12 which has been spread as a layer in wells and placed on a tray for placing in an oven in accordance with an embodiment of the present disclosure;

FIG. 14 is a picture of resulting dried film dosage form from FIG. 13, where the films have been peeled off the wells in accordance with an embodiment of the present disclosure;

FIG. 15A is a picture of a dried film dosage form from FIG. 14 which is deposited onto wax paper in accordance with an embodiment of the present disclosure;

FIG. 15B is a picture of a dried film which is deposited onto wax paper of FIG. 15A which is packaged in a sealable pouch, in accordance with an embodiment of the present disclosure;

FIG. 16A is a picture of dried films which are packaged deposited onto wax paper as in FIG. 15A and which packaged into a film holder box having a cover in accordance with an embodiment of the present disclosure;

FIG. 16B is a picture of dried films in their respective molds which are packaged into a film holder box containing slots to arrange molds and having a cover in accordance with an embodiment of the present disclosure.

In the drawings, exemplary embodiments are illustrated by way of example. It is to be expressly understood that the description and drawings are only for the purpose of illustrating certain embodiments and are an aid for understanding. They are not intended to be a definition of the limits of the invention.

### DETAILED DESCRIPTION

A detailed description of one or more embodiments of the invention is provided below along with accompanying figures that illustrate the principles of the invention. The invention is described in connection with such embodiments, but the invention is not limited to any embodiment. The scope of the invention is limited only by the claims. Numerous specific details are set forth in the following description in order to provide a thorough understanding of the invention. These details are provided for the purpose of non-limiting examples and the invention may be practiced according to the claims without some or all of these specific details. For the purpose of clarity, technical material that is known in the technical fields related to the invention has not been described in detail so that the invention is not unnecessarily obscured.

The present inventors have through R&D work developed a concentrated gel base composition which is useful for pharmaceutical compounding and surprisingly and unexpectedly is compoundable into a film strip dosage form and, upon dilution, into a gel dosage form. In other words, this concentrated gel base composition represents a flexible 2-in-1 compounding concentrated gel base composition, which can be designed into a mucoadhesive gel or film strip delivery form according to a particular application and/or medical needs of an individual patient.

Without being bound by any theory, the present inventors believe that the concentrated gel base composition of the present disclosure allows for at least a number of advantageous features. For example, the concentrated gel base composition of the present disclosure may present one or more of the following characteristics:
may afford more versatility than what is known to exist in the market in terms of sites of application. Such sites of application may include various administration routes, such as oral, ocular, vaginal, rectal and nasal. This characteristic may increase patient compliance since it may represent a more comfortable route and/or reduce active compound administration frequency;
may have a significantly higher viscosity than what is known to exist in the market, which affords the compounding personnel to dilute the base to the point of obtaining a desired viscosity, such as a more fluid gel or a spray;
may afford more versatility than what is known to exist in the market in terms of dosage forms. To the inventors' knowledge, mucoadhesive base products are marketed as being compoundable into a gel or a film strip but cannot be compounded into both dosage forms. The herein described 2-in-1 compounding concentrated gel base composition allows more flexibility to the compounding personnel to prepare a formulation which is appropriate to (or customized for) a given patient;
may avoid first-pass hepatic metabolism which translates into less drug degradation by the patient's organism before the drug can provide the desired clinical effects. First-pass hepatic metabolism is known to reduce oral dosage forms effectiveness in that part of the drug delivered through oral dosage forms will be degraded before it arrives on the site of action;
may provide systemic and local effect;
may increase the bioavailability of active compound since upon processing, the herein described flexible 2-in-1 compounding concentrated gel base composition can provide intimate contact with the absorption site.

In some embodiments, the film strip or gel dosage composition described herein is manufactured into a size that can advantageously allow its placement in the sublingual cavity allowing for potent, lipophilic molecules to be absorbed transmucosal with minimal saliva response and minimal swallowing of the drug. This avoidance of gastrointestinal (GI) uptake allows for a more rapid and consistent onset of action, more consistent plasma concentrations and higher bioavailability. This route of administration minimizes drug uptake via the GI route, which is variable and by which significant metabolism of the drug in the stomach and intestines can occur. In other words, by adhering to the oral mucosa during the period of drug delivery, the dosage form can minimize the saliva response and therefore can minimize delivery of the drug to the GI tract, such that the majority of drug is delivered across the oral mucosa. In such instances, the dosage form can be manufactured into a small volume dosage form that has a volume, for example but without being limited to, of from about 0 μl (microliters) to about 1000 μl and a mass of from about 0 mg (milligrams) to about 200 mg.

### Concentrated Base Composition

Generally speaking, the herein described concentrated gel base composition is a highly viscous composition. For example, the composition has a viscosity of at least 50,000 cPs, or of at least 80,000 cPs, or at least 100,000 cPs, or at least 200,000 cPs, or at least 300,000 cPs, or at least 500,000 cPs.

In one practical embodiment, the herein described concentrated base composition includes a blend of materials, which make it suitable for the herein described 2-in-1 flexible compounding application.

In some embodiments, the herein described base composition includes one or more mucoadhesive polymers, such as, natural, semisynthetic or synthetic polymers, which help in the systemic delivery of active ingredients (e.g., pharmaceutical, cosmetic, cannabinoid, nutraceutical, etc.).

Natural polymers may include, but without being limited to, amylopectin, zein, modified zein, casein, gelatin, serum albumin, collagen, chitosan, oligosaccharides and polysaccharides such as pyrrolidones, dextrins, cellulose, dextrans, tamarind seed polysaccharide, gellan, carrageenan gum, xanthan gum, Arabic gum, hyaluronic acid, polyhyaluronic acid, alginic acid or sodium alginate, locust bean gum, pullulan, poloxamers, maltodextrins, Eudragit™, guar gum, tragacanth gum, modified cellulose gum, and the like. It is to be noted that any of the herein listed compound can also include corresponding salts thereof, e.g., hyaluronic acids and salts thereof, alginic acids and salts thereof, carrageenans and salts thereof, and the like.

In one non-limiting embodiment, the herein described base composition includes mucoadhesive polymers, which are all natural mucoadhesive polymers.

In one non-limiting embodiment, the herein described base composition includes the following blend of polymers: carrageenan gum, xanthan gum, locust bean gum, and pullulan.

The herein described base composition can be processed to obtain, for example, an oral film product suitable for oral use and with a rapid dissolution when in contact with the oral mucosa. Without being bound by any theory, the present inventors have found that such herein described base composition makes the film product more elastic and cohesive, increases water retention, reduces syneresis (i.e., extraction or expulsion of a liquid from a gel) and produces a creamy-like texture oral tactile sensation (increase in patient compliance). This is believed to mainly occur because of interactions between the different gums in the formulation, which makes the gel thermoreversible and increases its viscosity.

Again, without being bound by any theory, the present inventors believe that the presence of pullulan in some embodiments of the herein described base composition may be advantageous in some instances in that this polymer exhibits mucoadhesivity, fast dissolution when in contact with salivary mucins, and a low penetration of both oxygen and water.

Again, without being bound by any theory, the present inventors believe that the presence of locust bean gum (ceratonia silique seed polysaccharides) in some embodiments of the herein described base composition may be advantageous in some instances in that this polymer exhibits swelling ability to afford a controlled release of the active ingredient.

The mucoadhesive polymer is typically present in the base gel composition described herein at 1-50% w/w, preferably 1-40% w/w or most preferably between 5-30% w/w. The base gel composition described herein may contain one or more different mucoadhesive polymer in any combination.

In some embodiments, the herein described base composition further includes one or more pharmaceutically acceptable polyhydric alcohols or mixtures thereof. Pharmaceutically acceptable polyhydric alcohols or mixtures thereof include, for example, mannitol, glucose, sucrose, dextrose, sorbitol, xylitol, maltitol and erythritol. Other water-soluble saccharides may also be employed, and combinations of water-soluble saccharides may be used. Without being bound by any theory, the present inventors have found that the presence of one or more polyhydric alcohols in some embodiments of the herein described base composition provides an advantageous pharmaceutical composition by virtue of its good stability and acceptable taste.

In some embodiments, the herein described base composition further includes one or more agents that operate to increase the strength and/or reduce the brittleness of the base composition when it is dried to a film dosage form. For example, such agents may include plasticizer agents such as glycerin, alkylene glycols, polyalkylene glycols, glycerol, triacetin, deacetylated monoglyceride, diethyl salate, triethyl citrate, dibutyl sebacate, polyethylene glycols, propylene glycol, castor oil, and the like.

In some embodiments, the herein described base composition further includes one or more suitable emulsifying agent such as poloxamers (e.g., poloxamer 407), benzalkonium chloride, polysorbates (Tween™ 20, Tween 80, etc.) and sodium lauryl sulfate, or opacifying agents such as titanium dioxide and the like. Without being bound by any theory, the present inventors believe that the presence of one or more suitable emulsifying agent (e.g., Tween 80) in some embodiments of the herein described base composition may facilitate solubilizing compounds into the base composition that would otherwise be more difficult to solubilize, such as poorly water-soluble compounds, therefore, addressing active ingredient compatibility problems seen in at least some delivery systems of the prior art which can often be limited to hydrophilic compounds.

The emulsifying agent is typically present in the base gel composition described herein at a concentration of from 0.01 to 3% weight percent of the composition. The base gel composition described herein may contain one or more different emulsifying agent in any combination.

In some embodiments, the base gel composition described herein and/or the processed dosage form described herein may further include one or more absorption enhancers, one or more buffering excipients and/or one or more coatings to improve, for example, hardness and friability.

In some embodiments, the base gel composition described herein and/or the processed dosage form described herein may further include one or more excipients that may affect both disintegration kinetics and drug release, and thus pharmacokinetics. Such additives may be selected from starch, carboxy-methycellulose type or crosslinked Polyvinyl Pyrrolidone (such as cross-povidone, PVP-XL), alginates, cellulose-based disintegrants (such as purified cellulose, methylcellulose, crosslinked sodium carboxy methylcellulose (Ac-Di-Sol) and carboxy methyl cellulose), microcrystalline cellulose (such as Avicel), ion exchange resins (such as Ambrelite IPR 88), gums (such as agar, locust bean, karaya, Pectin and tragacanth), guar gums, gum Karaya, chitin and chitosan, Smecta, gellan gum, Isapghula Husk, Polacrillin Potassium (Tulsion³³⁹) gas-evolving disintegrants (such as citric acid and tartaric acid along with the sodium bicarbonate, sodium carbonate, potassium bicarbonate or calcium carbonate), sodium starch glycolate (such as Explotab and Primogel), Addition of such additives may facilitate the fast break-up or disintegration of the dosage form into smaller particles that dissolve more rapidly than in the absence of such additives. An additional benefit of inclusion of such additives which contain the bioadhesive materials described herein, is that the smaller, drug-containing particles formed upon disintegration may have, by virtue of the highly increased surface area of contact with the oral mucosa, superior bioadhesive properties. In addition, the increased surface area may further facilitate the fast release of the active substance and thus further accelerate drug absorption and attainment of the required therapeutic levels systemically. Such additive may be used at a low level, typically 1-20% w/w relative to the total weight of the processed dosage form unit.

In some embodiments, the base gel composition described herein may include one or more preservatives such as potassium sorbate, sodium benzoate and the like.

### Active Ingredient

In one practical embodiment, the herein described concentrated gel base may be used for compounding one or more active ingredient(s) into a suitable mucoadhesive delivery dosage form.

In some embodiments, the one or more active ingredient(s) may include active pharmaceuticals ingredients (APIs), cosmetic ingredients, cannabinoids, nutraceuticals, and the like. Advantageously, the herein described base gel composition is compatible with compounding a broad range of active ingredients which can be lipophilic or hydrophilic ingredients.

The one or more active ingredient(s) can be present in any suitable and appropriate amount, depending upon the desired dosing. For example, in a 100 mg film strip, the active ingredient can be present in an amount of about 0.01-60 mg, about 0.1-50 mg, or about 0.5-40 mg.

Examples of “APIs” include, but are not limited to, anti-inflammatory, anesthetic, antiviral, anti-migraine, anti-emetic, antibiotics, analgesics, vaccines, anticonvulsants, antidiabetic agents, antifungal agents, antineoplastic agents, antiparkinsonian agents, anti-rheumatic agents, appetite suppressants, biological response modifiers, cannabinoid, cardiovascular agents, central nervous system stimulants, chemotherapy agents, contraceptive agents, dietary supplements, vitamins, minerals, lipids, saccharides, metals, amino acids (and precursors), nucleic acids and precursors, contrast agents, diagnostic agents, dopamine receptor agonists, nicotinic cholinergic receptor agonist (such as nicotine), erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroid, anti-hyperthyroid, anti-asthmatics, vertigo agents, and the like.

For example, the one or more active ingredient(s) may include a hormone replacement therapy (HRT) ingredient, such as feminine or masculine HRT ingredient(s). For example, HRT may be required in the case of postmenopausal women that frequently suffer from atrophic vaginitis or vulvar and vaginal atrophy (hereinafter “vulvovaginal atrophy” or “VVA”) with symptoms including, for example, vaginal dryness, vaginal odor, vaginal or vulvar irritation or itching, dysuria (pain, burning, or stinging when urinating), dyspareunia (vaginal pain associated with sexual activity), or vaginal bleeding associated with sexual activity, or other symptoms that may include soreness; with urinary frequency and urgency; urinary discomfort and incontinence (“estrogen-deficient urinary state(s)”), with one symptom of vaginal atrophy being an increased vaginal pH, which creates an environment more susceptible to infections. In such cases, the active ingredient may include 1 μg to about 25 μg of estradiol.

For example, the one or more active ingredient(s) may include one or more chemotherapeutic agents and inhibitors of membrane efflux systems to a systemic circulation for treatment, control and maintenance of cancer in a human. For example, existing systemic cancer therapy is almost exclusively limited to parenteral administration due to the barrier properties of the intestinal mucosa. Oral administration of chemotherapeutic agents prevents these agents to reach the systemic circulation in therapeutically relevant concentrations. Furthermore, oral administration of chemotherapeutic agents and inhibitors of membrane efflux systems, when attempted, often leads to significant gastrointestinal side effects such as acute nausea and vomiting, stomatitis, esophagitis, ulceration of stomach and colon, or increases risk of infections and/or toxic reactions as a result of reduced activity of membrane efflux systems in the alimentary and gastrointestinal mucosa. Extended or repeated parenteral administration of chemotherapeutic agents, as discussed above, has a potential to cause vascular collapse, vascular damage, phlebosclerosis, vascular hypersensitivities and other complications.

The vaginal route of delivery permits extended, continuous or pulsed delivery and administration of the drugs without need to visit the doctor's office or hospital. Using the mucosal composition and intravaginal device of the invention, the length of the drug delivery can be extended and the delivered dose may be lowered as the vaginal delivery by-passes the gastrointestinal tract and eliminates the intravenous administration with all its adverse effects and requirements. This is where the herein described base gel composition can also be useful in order to compound one or more chemotherapy agents into a film strip or gel dosage form for insertion into the vaginal cavity and adhesion to the vaginal mucosa.

Examples of “cosmetic” or “nutraceuticals” ingredients include, but are not limited to, breath freshening compounds like menthol, other flavors or fragrances, especially those used for oral hygiene, as well as actives used in dental and oral cleansing such as quaternary ammonium bases. The effect of flavors may be enhanced using flavor enhancers like tartaric acid, citric acid, vanillin, or the like. Anti-tartar agents for dental use may also be employed. Other examples may include Vitamin D, Resveratrol, melatonin, Coenzyme Q10, Biotin, Cyanocobalamin (Vitamin B12), Chromium polynicotinate, Folic acid, NADH (Nicotinamide Adenine Dinucleotide, Phytomenadione (Vitamin K1), and the like.

Examples of a “cannabinoid” include, but are not limited to, cannabichromanon (CBCN), cannabichromene (CBC), cannabichromevarin (CBCV), cannabicitran (CBT), cannabicyclol (CBL), cannabicyclovarin (CBLV), cannabidiol (CBD), cannabidiol monomethylether (CBDM), cannabidiol-C4 (CBD-C4), cannabidiorcol (CBD-C1), cannabidiphorol (CBDP), cannabidivarin (CBDV), cannabielsoin (CBE), cannabifuran (CBF), cannabigerol (CBG), cannabigerol monomethylether (CBGM), cannabigerolic acid (CBGA), cannabigerovarin (CBGV), cannabinodiol (CBND), cannabinodivarin (CBVD), cannabinol (CBN), cannabinol methylether (CBNM), cannabinol propyl variant (CBNV), cannabinol-C2 (CBN-C2), cannabinol-C4 (CBN-C4), cannabiorcol (CBN-C1), cannabiripsol (CBR), cannabitriol (CBO), cannabitriolvarin (CBTV), cannabivarin (CBV), dehydrocannabifuran (DCBF), Δ7-cis-iso tetrahydrocannabivarin, tetrahydrocannabinol (THC), Δ9-tetrahydrocannabionolic acid B (THCA-B), Δ9-tetrahydrocannabiorcol (THC-C1), tetrahydrocannabivarinic acid (THCVA), tetrahydrocannabivarin (THCV), ethoxy-cannabitriolvarin (CBTVE), trihydroxy-Δ9-tetrahydrocannabinol (triOH-THC), 10-ethoxy-9hydroxy-Δ6a-tetrahydrocannabinol, 8,9-dihydroxy-Δ6a-tetrahydrocannabinol, 10-oxo-Δ6a-tetrahydrocannabionol (OTHC), 3,4,5,6-tetrahydro-7-hydroxy-α-α-2-trimethyl-9-n-propyl-2,6-methano-2H-1-benzoxocin-5-methanol (OH-iso-HHCV), Δ6a,10a-tetrahydrocannabinol (Δ6a,10a-THC), Δ8-tetrahydrocannabivarin (Δ8-THCV), Δ9-tetrahydrocannabiphorol (Δ9-THCP), Δ9-tetrahydrocannabutol (Δ9-THCB), derivatives of any thereof, and combinations thereof. Further examples of suitable cannabinoids are discussed in at least PCT Patent Application Pub. No. WO2017/190249 and U.S. Patent Application Pub. No. US2014/0271940, which are incorporated by reference in their entirety.

Cannabidiol (CBD) means one or more of the following compounds: Δ2-cannabidiol, Δ5-cannabidiol (2-(6-isopropenyl-3-methyl-5-cyclohexen-1-yl)-5-pentyl-1,3-benzenediol); Δ4-cannabidiol (2-(6-isopropenyl-3-methyl-4-cyclohexen-1-yl)-5-pentyl-1,3-benzenediol); Δ3-cannabidiol (2-(6-isopropenyl-3-methyl-3-cyclohexen-1-yl)-5-pentyl-1,3-benzenediol); Δ3,7-cannabidiol (2-(6-isopropenyl-3-methylenecyclohex-1-yl)-5-pentyl-1,3-benzenediol); Δ2-cannabidiol (2-(6-isopropenyl-3-methyl-2-cyclohexen-1-yl)-5-pentyl-1,3-benzenediol); Δ1-cannabidiol (2-(6-isopropenyl-3-methyl-1-cyclohexen-1-yl)-5-pentyl-1,3-benzenediol); and Δ6-cannabidiol (2-(6-isopropenyl-3-methyl-6-cyclohexen-1-yl)-5-pentyl-1,3-benzenediol). In a preferred embodiment, and unless otherwise stated, CBD means Δ2-cannabidiol.

Tetrahydrocannabinol (THC) means one or more of the following compounds: Δ8-tetrahydrocannabinol (Δ8-THC), Δ9-cis-tetrahydrocannabinol (cis-THC), Δ9-tetrahydrocannabinol (Δ9-THC), Δ9-tetrahydrocannabinolic acid A (THCA-A), Δ10-tetrahydrocannabinol (Δ10-THC), Δ9-tetrahydrocannabinol-C4, Δ9-tetrahydrocannabinolic acid-C4 (THCA-C4), synhexyl (n-hexyl-Δ3THC). In a preferred embodiment, and unless otherwise stated, THC means one or more of the following compounds: Δ9-tetrahydrocannabinol and Δ8-tetrahydrocannabinol.

The cannabinoid in the present disclosure may be in an acid form or a non-acid form, the latter also being referred to as the decarboxylated form since the non-acid form can be generated by decarboxylating the acid form. Preferably, where reference is made to a specific cannabinoid, it will be understood that the cannabinoid is in the decarboxylated form.

The cannabinoid in the compositions of the present disclosure may be a single cannabinoid or may be a combination of two or more cannabinoids. In a non-limiting example, the cannabinoid in the compositions of the present disclosure is cannabidiol (CBD), tetrahydrocannabinol (THC), or a mixture thereof.

As is known in the art, various cannabinoids can be used in combination to achieve a desired effect in a user. Suitable mixtures of cannabinoids that can be used in the present disclosure include but are not limited to a mixture of tetrahydrocannabinol (THC), and cannabidiol (CBD). Certain specific ratios of cannabinoids may be useful to produce the feeling of physical and/or emotional satisfaction and/or may be useful in the treatment or management of specific diseases or conditions.

In some embodiments, the (w/w) ratio of the THC to the CBD is between about 1:1000 and about 1000:1. Preferably, the (w/w) ratio of THC to CBD in the composition may be about 1:1000, about 1:900, about 1:800, about 1:700, about 1:600, about 1:500, about 1:400, about 1:300, about 1:250, about 1:200, about 1:150, about 1:100, about 1:90, about 1:80, about 1:70, about 1:60, about 1:50, about 1:45, about 1:40, about 1:35, about 1:30, about 1:29, about 1:28, about 1:27, about 1:26, about 1:25, about 1:24, about 1:23, about 1:22, about 1:21, about 1:20, about 1:19, about 1:18, about 1:17, about 1:16, about 1:15, about 1:14, about 1:13, about 1:12, about 1:11, about 1:10, about 1:9, about 1:8, about 1:7, about 1:6, about 1:5, about 1:4.5, about 1:4, about 1:3.5, about 1:3, about 1:2.9, about 1:2.8, about 1:2.7, about 1:2.6, about 1:2.5, about 1:2.4, about 1:2.3, about 1:2.2, about 1:2.1, about 1:2, about 1:1.9, about 1:1.8, about 1:1.7, about 1:1.6, about 1:1.5, about 1:1.4, about 1:1.3, about 1:1.2, about 1:1.1, about 1:1, about 1.1:1, about 1.2:1, about 1.3:1, about 1.4:1, about 1.5:1, about 1.6:1, about 1.7:1, about 1.8:1, about 1.9:1, about 2:1, about 2.1:1, about 2.2:1, about 2.3:1, about 2.4:1, about 2.5:1, about 2.6:1, about 2.7:1, about 2.8:1, about 2.9:1, about 3:1, about 3.5:1, about 4:1, about 4.5:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 35:1, about 40:1, about 45:1, about 50:1, about 60:1, about 70:1, about 80:1, about 90:1, about 100:1, about 150:1, about 200:1, about 250:1, about 300:1, about 400:1, about 500:1, about 600:1, about 700:1, about 800:1, about 900:1.

The compositions of the present disclosure may comprise the at least one cannabinoid in a concentration of from about 0.001 mg/mL to about 100 mg/mL, including any amount therebetween or any ranges therein; in a non-limiting example, the compositions may comprise from about 0.002 mg/mL to about 100 mg/mL, from about 0.1 mg/mL to about 75 mg/mL, or from about 0.1 mg/mL to about 50 mg/mL, including any amount therebetween or any ranges therein, of the at least one cannabinoid. Cannabinoids provided at such an amount in the compositions of the present disclosure can be particularly effective in penetrating the mucosa into the systemic circulation, preferably without having adverse side-effects.

The one or more active ingredient(s) may be found in the form of one or more pharmaceutically acceptable salts, esters, derivatives, analogs, prodrugs, and solvates thereof. In certain embodiments, the herein described one or more active ingredient(s) (e.g., API, cosmetic, cannabinoid, nutraceutical, etc.) is a poorly water-soluble compound (more or less hydrophobic).

Compounding Base Gel into a Gel Dosage or Film Dosage Form

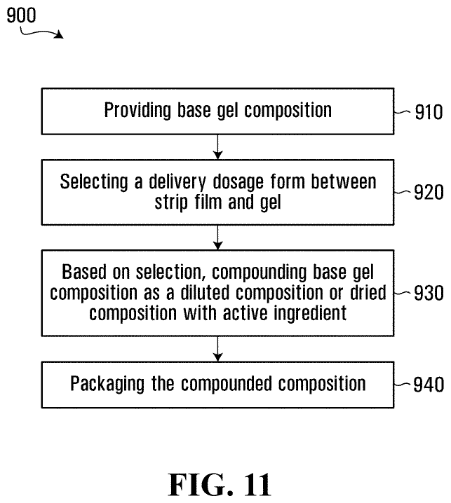

FIG. 11 is a block flow diagram illustrating a process 900 of using the herein described gel base composition for compounding a mucoadhesive formulation. For the purpose of the present disclosure, the expression “compounding” refers in particular to those single compositions which are assembled in a medical facility, or by a licensed pharmacy (as opposed to those compositions made in batch in a pharmaceutical industrial plant) where a pharmacist combines, mixes, or alters ingredients in response to a doctor's prescription to create a medicine tailored to the medical needs of an individual patient. In other words, the type and/or concentration of at least one of an active ingredient, excipient, diluent or carrier is customized to create a composition tailored to the medical needs of the patient.

Compounding may, thus, be used in a variety of situations where a patient cannot be treated with a standard, commercially available, FDA- (or other regulatory body) approved medicine.

For example, a patient might be allergic to the kind of dye used in a commercially available medication. In this case, the compounding personnel would formulate the medication without the dye or with another dye. Or, sometimes elderly patients or children who cannot swallow tablets need their medicine in a liquid or suppository form that is not commercially available. Suspensions possess certain advantages over other dosage forms. Some drugs are insoluble in all acceptable media and must, therefore, be administered as a tablet, capsule, or as a suspension. Because of their liquid character, suspensions represent an ideal dosage form for patients who have difficulty swallowing tablets or capsules. This factor is of particular importance in administration of drugs to children. Suspensions of insoluble drugs may also be used externally, often as protective agents.

In addition, disagreeable tastes can be masked by a suspension of the drug or a derivative of the drug, an example of the latter being the drug chloramphenicol palmitate. Finally, drugs in suspension are chemically more stable than in solution. This is particularly important with certain antibiotics and the pharmacist is often called on to prepare such a suspension just prior to the dispensing of the preparation.

Sometimes, a patient may require a special active ingredient dosage and thus, the compounding personnel will customize the active ingredient concentration in the compounded composition.

In other cases, a patient may be allergic to the active ingredient in the commercially available medication and the compounding personnel will thus customize the composition by replacing the active ingredient with another one, hypoallergenic for the patient.

The person of skill will recognize that such are examples of a composition which is personalized for a patient.

Returning to the process 900, this process includes a first step 910 where the compounding personnel is provided with the gel base composition. The gel base composition can be packaged in, for example, a jar, a pouch, or any other suitable container. In step 920, the compounding personnel can select to process at least a portion of such gel base composition in order to obtain a gel dosage form or a film strip dosage form. As further described herein, the selectively aspect depends on the desired compounding application, such as patient requirement, site of administration, active ingredient(s), delivery rate, human or veterinary applications, and the like. For example, the selection can be based on an instruction set out in the prescription prepared by the medical personnel or may take the form of any other type of instruction.

Independently of the dosage form selected, if the active ingredient is a particle, the active ingredient may be incorporated in dry powder form, or in solution (where soluble), suspension or emulsion. One advantage of dry powder form is to avoid the time during which the active ingredient is in contact with liquid (which can avoid problems with crystallization, drug stability, and the degradation of taste masking or controlled release systems).

### Selectively Opting to Obtain the Gel Dosage Form

In step 930, when the compounding personnel selectively opts for obtaining the gel dosage form, processing may include, for example, diluting the gel base with a diluting agent to obtain a target viscosity. Dilution with the diluting agent can occur, prior to, during or after incorporating an active ingredient therein (e.g., a pharmaceutical, cosmetic, cannabinoid, nutraceutical, etc.).

Alternatively or additionally, in step 930, when the compounding personnel selectively opts for obtaining the gel dosage form, processing may include, for example, diluting the gel base with a diluting agent to obtain a gel having a target maximum compressive force (MCF)≤5.0 and a maximum adhesive force (MAF) ≥2.0. The MCF and MAF can be measured using a TA-XT2i HR texture analyzer (Stable Micro Systems, United Kingdom).

The diluting agent can be, for example, an acceptable carrier, excipient or diluent (e.g., purified water or an aqueous composition). The diluting agent may be a liquid, a cream or another gel compatible for being in contact with mucosal surfaces (e.g., VersaPro™ Gel and Cream Base, simple syrup, and the like).

The target viscosity may be determined based on a particular application and/or on desired mucosa coating properties.

The base gel composition can be diluted to a target viscosity of, for example but without being limited to, less than 40,000 cPs. For example, a target viscosity in the range of 500 to 40,000 cPs affords a gel, which can be suitable for forming a vaginal gel dosage form; diluting the composition to a target viscosity of, for example but without being limited to, 100 to 1000 cPs affords a gel, which can be suitable for forming a rectal enema form; diluting the composition to a target viscosity of, for example but without being limited to, 500 to 40000 cPs affords a gel, which can be suitable for forming an oral/dental gel form.

In one embodiment, the herein described gel dosage form is a hydrogel dosage form.

### Selectively Opting to Obtain the Film Dosage Form

Returning to the case when the compounding personnel selectively opts for obtaining the film strip dosage form, step 930 may include, for example, incorporating the active ingredient (e.g., pharmaceutical, cosmetic, cannabinoid, nutraceutical, etc.) in the gel base composition to obtain a mixture, spreading the mixture in the form of a layer on a suitable substrate and drying the mixture to obtain the desired film strip dosage form. Optionally, the gel base composition can be diluted with a diluting agent prior to, during or after incorporating the active ingredient therein (e.g., a pharmaceutical, cosmetic, cannabinoid, nutraceutical, etc.).

The step of spreading the mixture may be performed using (i) automatic continuous film making equipment, (ii) a manual or automatic spreader, or (iii) molds of specified dimensions and shapes (e.g., in single units or multiple unit blocks). Film casting process and technology, dosage flexibility required for different patient categories, cost of the equipment, space requirements, complexity of process, material waste, set-up or cleaning time and packaging difficulties associated with the first two processes of making medicated films in the compounding pharmacy set up may not be ideal in certain circumstances. The alternative method to overcome those constraints would be using the film casting in prefabricated molds.

The process for manufacturing the film strip dosage form includes a drying step. The drying step may include, for example, submitting the mixture to a thermal treatment at a temperature of about 30° C. to about 70° C., preferably about 40° C. to about 60° C., for a pre-determined time, which is sufficient to obtain the desired film strip dosage form having appropriate physical flexibility. For example, the pre-determined time may include a time selected from about 30 to about 120 min, such as about 30 minutes. For example, the thermal treatment may be performed using a convection oven or any other drying chamber (UV/IR/Microwave/electronic heat source/heat tunnel) equipped with air circulation and/or vacuum pump. The drying step may include an additional air-drying step at room temperature (e.g., about 22° C.) either under a hood or in open air (e.g., on the bench) for a period of time of from about 15 to about 120 minutes, the latter being feasible provided that relative humidity of ambient air is below 45%, preferably <40%.

It will be apparent to the person of skill that, in some embodiments, the active ingredient can be incorporated into the composition prior to, during or after the drying step. For example, the above procedure involves incorporating the active ingredient prior to the drying step. In another embodiment, the active ingredient can be incorporated by infusing the film with the active ingredient during or after the drying step or the above-discussed additional air-drying step.

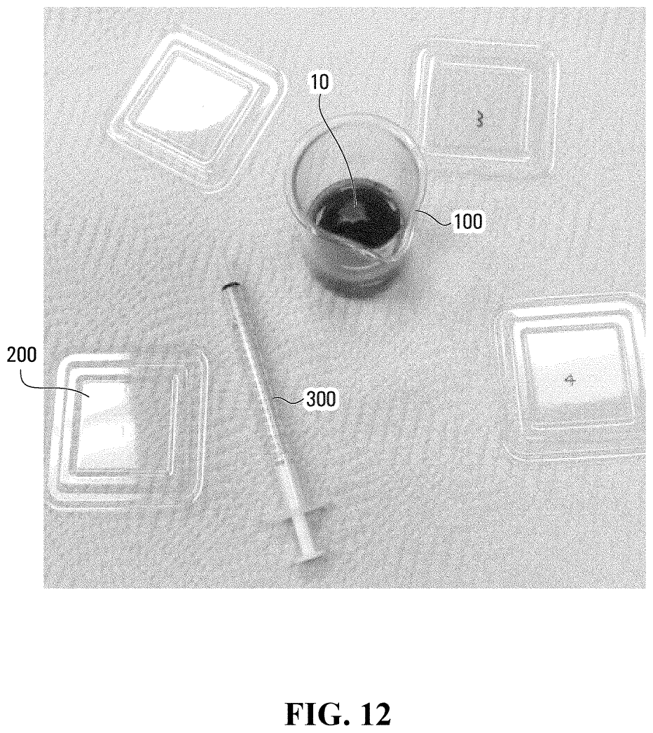

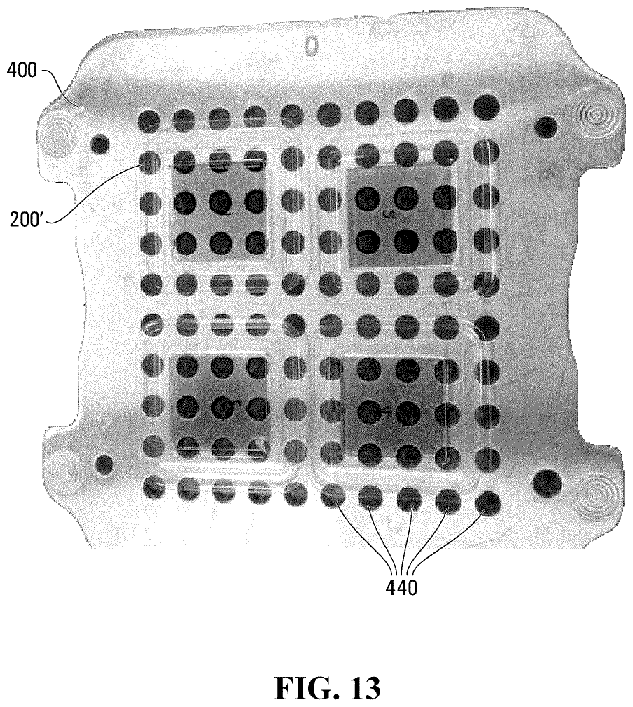

In a practical implementation, the film strip can be manufactured using prefabricated molds as per the following procedure. In reference to FIG. 12, the composition 10 described herein is first homogeneously blended in a container 100 with an active pharmaceutical ingredient (API) and optional excipients (e.g., levigating agent, color, flavor, sweetener, etc.) as required to obtain a substrate gel/solution. Measured quantity of the substrate gel/solution are then layered onto a suitable substrate, e.g., into a film casting mold 200 that includes a cavity well 240 using a suitable metered delivering device 300 (e.g., PreciseDose™ dispenser, automatic or any electronic dispensing device, etc.). The substrate gel/solution is uniformly spread in the mold cavity with syringe tip, mechanical vibration, or any other form of electronic, mechanical or manual device depending upon the viscosity of the substrate gel/solution to obtain a substrate gel-containing mold 200′, as best shown in FIG. 13. One or more substrate gel-containing mold(s) 200′ are placed on a base plate 400 (e.g., made of glass, metal, or heat resistant plastic) and held in a convection oven or any other drying chamber (not shown), e.g., UV, IR, microwave, electronic heat source, or heat tunnel, equipped with air circulation and/or vacuum pump. The substrate gel-containing mold(s) 200′ are held at specified temperature (e.g., from about 30° C. to about 70° C.) for a specified time (e.g., from about 30 to about 120 min) to evaporate the liquid and consequently result in a dried film 500.

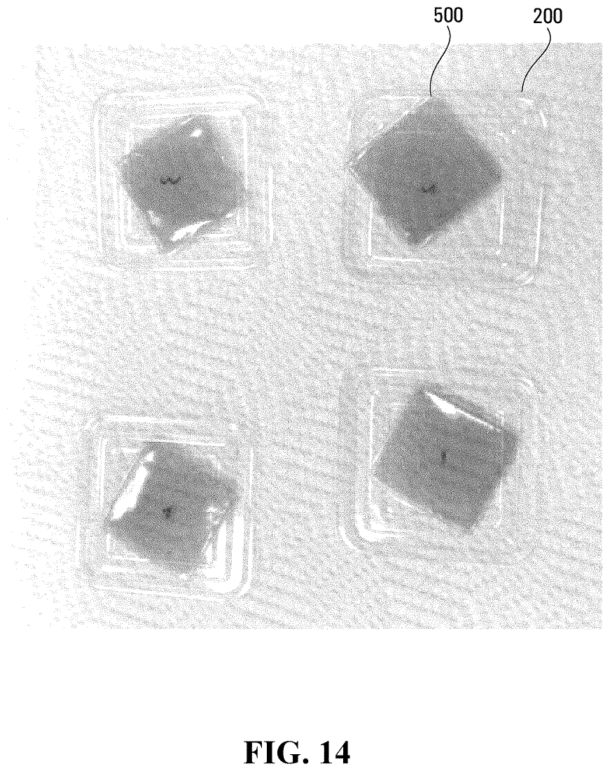

Preferably, the base plate 400 holding the molds 200′ is on an evenly leveled surface, preferably with a plurality of perforations 440 for uniform drying and even thickness of the film. On completion of drying, the substrate gel-containing mold(s) 200′ are taken out from the drying chamber or oven and allowed to attain room temperature. The resulting dried films 500 are then peeled off from the one or more molds 200 as shown in FIG. 14 and, if required, kept at the room temperature and controlled humidity (e.g., <45%, preferably <40%) for an additional 15-120 minutes for air-drying.

For example, the prefabricated molds 200 can be made with thin flexible pharma grade, heat stable material (e.g., polyethylene (PE)/polyethylene terephthalate (PET)/aluminum/polyvinyl chloride (PVC)/polyvinylidene dichloride (PVDC), and the like). For example, the prefabricated molds can be made with defined dimensions and depth as the base for film casting base. The well or mold will generally be of suitable depth to contain the wet height of the desired film composition. As a practical matter, the film tends to reduce in thickness when dried. The well or mold may be pretreated with silicone, hydrophobic agents, or and other suitable material that promotes flow of the film composition and/or promotes release of the final dry film from the well or mold. Round shapes of the well or mold are desirable, but non limiting, to form a round film. Circular shapes may be particularly desirable, but square or rectangular forms are also possible. In the case of circular shapes, the film composition can be deposited in the center and flow outward. Any regular or irregular polygonal shape may also be possible. The mold or well may also be shaped for form three dimensional attributes on the bottom of the film.

The sides of the well or mold may be perpendicular, angled (outward from the planar bottom surface). In certain embodiments, the well or mold is sufficiently flexible to allow a consumer to readily push the bottom of the well or mold up to present the film for easy access by the patient. The well or mold material must be able to withstand drying temperatures of the drying process.

In most embodiments, the area of the mold or well defines the dimensions of the film. The mold or well is of fixed size. Generally, the mold or well with have a deposit surface of 9 square inches or less. Larger size are possible, smaller sizes are preferred for comfort (in oral use), as well as to speed flow of the film composition to ultimate dimensions (i.e. a shorter distance to travel).

In certain embodiments, the molds or wells are fixed, and the dried films 500 are removed from said molds or wells which can then be reused.

In some embodiments, the dried film strip 500 can have a thickness of about 0.01 mm to about 20 mm, for example of from about 0.0254 mm (1 mils) to about 1.016 mm (40 mils). Thickness may be augmented above the foregoing thicknesses, including without limitation, for dermal use of the end product.

In some embodiments, the drying is performed to obtain a water activity (a_{w}) of ≤0.6, such as ≤0.5, ≤0.4, ≤0.3, ≤0.2, ≤0.1, and the like (e.g., 0.04≤a_{w}≤0.6). Water activity (a_{w}) is a measurement of the availability of water for biological reactions. It determines the ability of microorganisms to grow. If water activity decreases, microorganism's ability to grow will also decrease. Water activity is a useful measure of microbial stability that can be successfully used in product development and microbial risk assessments to support specification setting and testing decisions for both product release and stability studies Water activity may be measured according to materials and procedures known in the art, for example, using an Aqualab Water Activity Meter 4TE (Decagon Devices, Inc., U.S.A.).

Once the film is dry, it is possible to print or emboss an identifier on the film dosage form. For printing, individual print heads are typically required. Printing can be targeted and calibrated in a precise location on the film. Also, the well may have a chevron such that it leaves an imprint identity on the dried film.

Multi-layer films are possible simply by addition depositions of active ingredients, or additional film compositions in the manufacturing process. For example, a semi insoluble (or insoluble) backing layer may be separately deposited on a deposited film. This may be done after the first layer is dried, or where density and miscibility will permit separate deposit on non-dried layers, on a non-dried layer. Similarly, a special layer of muco-adhesive, permeation enhancers, or other excipients disclosed herein may be deposited separately where desired.

### Packaging

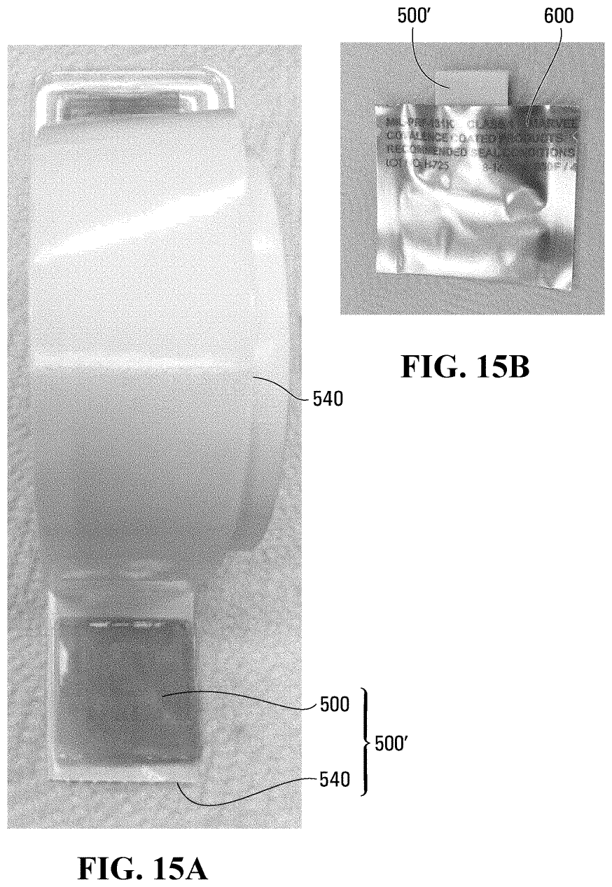

Finally, in step 940, the compounded composition in either film strip or gel dosage composition can be packaged into a suitable packaging, such as for example a jar, pouch, foil, and the like. In the case of strip film dosage form, the individual dried films 500 can be wrapped with or deposited onto wax paper 540 (where in the latter case, the wax paper may function as a support for the film) to obtain wrapped dry films 500′. The required quantity of films packaged into sealable aluminum or polyethylene (PE) pouch 600 for dispensing, as shown in FIG. 15A and FIG. 15B.

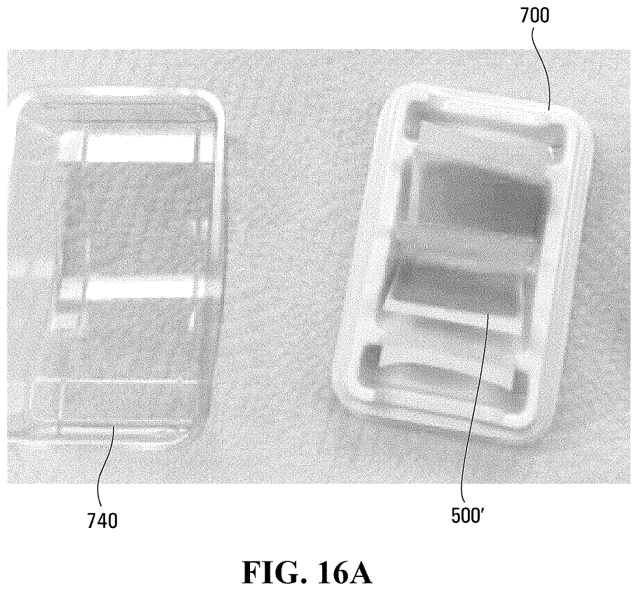

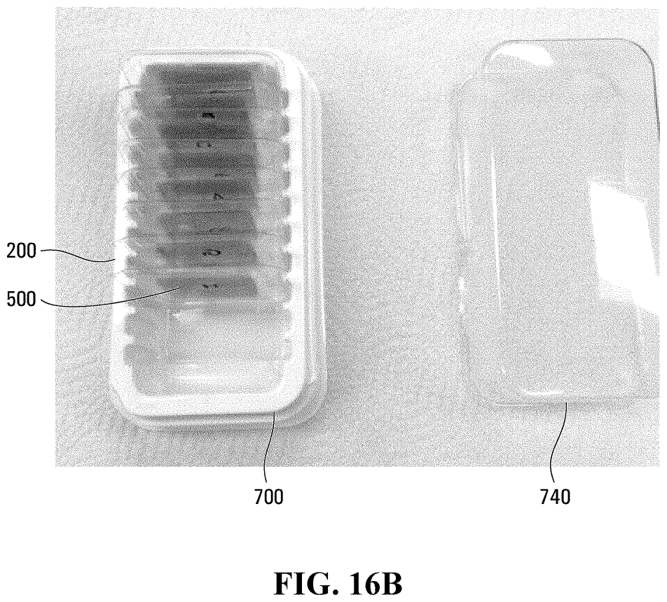

Alternatively, the whole mold 200 containing the dried films 500, or the wrapped dry films 500′ can be packaged into a PE film holder box 700 containing slots to arrange dried films or molds, as shown in respective FIGS. 16A and 16B. Optionally, the PE film holder box 700 may be closed with a cover 740. The box 700 with or without cover 740 can be sealed in an aluminum or PE pouch (not shown). Alternatively or additionally, the box 700 can be packaged in blister/bubble packing.

It will be apparent that in such embodiments, the film casting process using prefabricated molds is simple and practical; this becomes an appropriate choice for making medicated films for oral and topical applications in the compounding pharmacy setting.

The final package may be child resistant. The molds or wells may be joined together in a group of packages or may be separated. Such separation may occur at the time of filling/deposit or later, i.e. after drying.

### Test Procedures

### Compressive Test

The compressive test consists of determining a diluted gel's response to an applied force (stress) or deformation (strain) and assess textural quality and freshness. The test set up requires providing a container, such as a cup, and loading a volume of the gel therein and testing the compressive characteristics of the gel using a suitable device, such as for example a TA-XT2i HR texture analyzer (Stable Micro Systems, United Kingdom). The test procedure is as follows:
1) Providing a set of gel samples to be tested. The gel samples are all made in a single batch or individually but in a sufficiently controlled environment such as to ensure that a high degree of uniformity between the gel samples is provided.
2) A sufficient quantity of the gel is scooped into the container to create the desired test sample depth. For example, each gel is scooped into a ˜39 mm diameter plastic cup until a depth of about 20 mm is reached.
3) Testing is performed at ambient temperature (e.g., 20° C.) and humidity (e.g., 40%) and is accomplished using a ½″ diameter stainless steel probe with a 1″ radius of curvature.
4) The gel sample is deformed to a depth of 15 mm using a probe speed of 2 mm/s, held at the 15 mm depth for 1 second, and then removed at 2 mm/s.
5) After waiting 15 seconds (and without cleaning the probe) a second deformation was performed; these two deformations are referred to as A and B, respectively, for a given trial.
6) Determine a load-time curve for the test gel. From this curve, a maximum compressive force (MCF, observed during the indentation portion of deformation) and a maximum adhesive force (MAF, observed during the probe removal portion of deformation) can be determined. The test is repeated at least 3 times to obtain an average value.

Note that for the purpose of the present description, the above defined test procedure will be referred to as a “compressive test”.

In one aspect of the present disclosure, the gel obtained by diluting the gel base composition has an average MCF ≤−3.0 g, or ≤−3.5 g, or ≤−4.0 g, or ≤−4.5 g, or ≤−5.0 g, or ≤−5.5 g, or ≤−6.0 g, or ≤−6.5 g, or ≤−7.0 g, or ≤−7.5 g, or ≤−8.0 g, or ≤−8.5 g, or ≤−9.0 g, or ≤−9.5 g.

In one aspect of the present disclosure, the gel obtained by diluting the gel base composition has an average MAF ≥1.8 g, or ≥2.0 g, or ≥2.5 g, or ≥3.0 g, or ≥3.5 g.

In one aspect of the present disclosure, the gel obtained by diluting the gel base composition has an average MCF ≤−3.0 g, or ≤−3.5 g, or ≤−4.0 g, or ≤−4.5 g, or ≤−5.0 g, or ≤−5.5 g, or ≤−6.0 g, or ≤−6.5 g, or ≤−7.0 g, or ≤−7.5 g, or ≤−8.0 g, or ≤−8.5 g, or ≤−9.0 g, or ≤−9.5 g, and an average MAF ≥1.8 g, or ≥2.0 g, or ≥2.5 g, or ≥3.0 g, or ≥3.5 g.

### Tensile Test

The tensile test consists of determining a film strip's response to a pulling force. The test is a variant of ASTM D882 test which is the Standard Test Method for Tensile Properties of Thin Plastic Sheeting and of ASTM D638 test whereby plastic material is pulled until in breaks in order to measure elongation, tensile modulus, tensile yield strength, and tensile strength at break. The test can be performed using an ADMET eXpert 7601 Universal Testing System or the RSA G2 rheometer. The test procedure is as follows:
1) Providing a set of strip film samples to be tested. The strip film samples are all made in a single batch or individually but in a sufficiently controlled environment such as to ensure that a high degree of uniformity between the strip film samples is provided.
2) Cutting the strips using a ½″ wide die. The width and thickness of the strip film samples must be measured and averaged as quickly as possible to minimize the time the strip samples are exposed to ambient conditions prior to testing. The strips are kept in their sealed pouches until immediately prior to testing.
3) Data is collected using a 0.05 mm/s crosshead speed and a grip-to-grip separation of 1 cm.
4) Determine a load-deflection curve for a test strip sample and obtain the maximum load (at break) and corresponding stress for each sample. Repeat the test with at least 5 samples.

In one aspect of the present disclosure, the dried film is characterized with an average load at break ≥1000 g, or ≥1050 g, or ≥1100 g, or ≥1150 g, or ≥1200 g, or ≥1250 g, ≥1300 g, ≥1350 g, or ≥1400 g.

In one aspect of the present disclosure, the dried film is characterized with an average stress at break of ≥10.0×10⁷ Dynes/cm², or ≥11.0×10⁷ Dynes/cm², or ≥12.0×10⁷ Dynes/cm², or ≥13.0×10⁷ Dynes/cm², or ≥14.0×10⁷ Dynes/cm², or ≥15.0×10⁷ Dynes/cm², or ≥16.0×10⁷ Dynes/cm², or ≥17.0×10⁷ Dynes/cm², or ≥18.0×10⁷ Dynes/cm², or ≥19.0×10⁷ Dynes/cm², or ≥20.0×10⁷ Dynes/cm².

In one aspect of the present disclosure, the dried film is characterized with an average load at break ≥1000 g, or ≥1050 g, or ≥1100 g, or ≥1150 g, or ≥1200 g, or ≥1250 g, ≥1300 g, ≥1350 g, or ≥1400 g, and an average stress at break of ≥10.0×10⁷ Dynes/cm², or ≥11.0×10⁷ Dynes/cm², or ≥12.0×10⁷ Dynes/cm², or ≥13.0×10⁷ Dynes/cm², or ≥14.0×10⁷ Dynes/cm², or ≥15.0×10⁷ Dynes/cm², or ≥16.0×10⁷ Dynes/cm², or ≥17.0×10⁷ Dynes/cm², or ≥18.0×10⁷ Dynes/cm², or ≥19.0×10⁷ Dynes/cm², or ≥20.0×10⁷ Dynes/cm².

### Definitions

Unless otherwise defined, all technical and scientific terms used herein have the same meaning as commonly understood by a person of ordinary skill in the art to which the present invention pertains. As used herein, and unless stated otherwise or required otherwise by context, each of the following terms shall have the definition set forth below.

For the purpose of the present disclosure, the expression “hydrogel” refers to a polymer network that can be extensively swollen with water. Hydrophilic gels that are usually referred to as hydrogels are networks of polymer chains that are sometimes found as colloidal gels in which water is the dispersion medium. Researchers, over the years, have defined hydrogels in many different ways. The most common of these is that hydrogel is a water-swollen, and cross-linked polymeric network produced by the simple reaction of one or more monomers. Another definition is that it is a polymeric material that exhibits the ability to swell and retain a significant fraction of water within its structure but will not dissolve in water. The reader is invited to read the scientific review article of Ahmed, E M, Journal of Advanced Research, Volume 6, Issue 2, March 2015, Pages 105-121 for more detailed information in hydrogels.

For the purpose of the present disclosure, the expression “pullulan” refers to a linear, water soluble polysaccharide polymer consisting of maltotriose units connected to each other by an α-1,6 glycosidic bond. The three glucose units in each maltotriose unit are connected by an α-1,4 glycosidic bond. The linkage pattern of pullulan is responsible for the adhesive properties of the polysaccharide and its capacity for forming fibers and oxygen-impermeable films. Pullulan is typically produced from starch by the fungus Aureobasidium pullulans and can be produced commercially by batch fermentation as described in Leathers, Appl. Microbiol. Biotechol., 62:468-473 (2003).

For the purpose of the present disclosure, the expression “polyol” refers to an organic compound containing multiple hydroxyl groups, such as sugar alcohols. Examples of polyols which may be useful in the context of the present invention may include mannitol, maltitol, sorbitol, xylitol, erythritol, and isomalt.

As used herein, when a composition is said to “adhere” to a surface, such as a mucosal membrane, it is meant that the composition is in contact with the surface and is retained upon the surface without the application of an external force. Adhesion is not meant to imply any particular degree of sticking or bonding, nor is it meant to imply any degree of permanency.

For the purpose of the present disclosure, the expression “Tensile strength” refers to the maximum stress that a material can withstand while being stretched or pulled before failing or breaking. Tensile strength is the opposite of compressive strength and the values can be quite different. Tensile strength is defined as a stress, which is measured as force per unit area. For some non-homogeneous materials (or for assembled components) it can be reported just as a force or as a force per unit width. In the SI system, the unit is the pascal (Pa) (or a multiple thereof, often megapascals (MPa), using the mega-prefix); or, equivalently to pascals, newtons per square meter (N/m²). The customary unit is pounds-force per square inch (lbf/in² or psi), or kilo-pounds per square inch (ksi, or sometimes kpsi), which is equal to 1000 psi; kilo-pounds per square inch are commonly used for convenience when measuring tensile strengths. Typically, the testing involves taking a small sample with a fixed cross-section area, and then pulling it with a controlled, gradually increasing force until the sample changes shape or breaks.

For the purpose of the present disclosure, the expression “carrier” describes a material that does not cause significant irritation to an organism and does not abrogate the biological activity and properties of the compound of the composition described herein. Carriers must be of sufficiently high purity and of sufficiently low toxicity to render them suitable for administration to the mammal being treated. The carrier can be inert, or it can possess pharmaceutical benefits. Carriers and vehicles useful herein include any such materials know in the art which are nontoxic and do not interact with other components. The carrier can be liquid or solid and is selected, with the planned manner of administration in mind, to provide for the desired bulk, consistency, etc., when combined with an active agent and other components of a given composition.

For the purpose of the present disclosure, the term “plasticizer” refers to a material that, when added to a polymer, imparts an increase in flexibility, workability, and other properties to the finished product.

As used herein, a “pharmaceutically acceptable salt” is understood to mean a compound formed by the interaction of an acid and a base, the hydrogen atoms of the acid being replaced by the positive ion of the base. Non-limiting examples of pharmaceutically acceptable salts include sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate. Another method for defining the ionic salts may be as an acidic functional group, such as a carboxylic acid functional group, and a pharmaceutically acceptable inorganic or organic base. Non-limiting examples of bases include, but are not limited to, hydroxides of alkali metals such as sodium, potassium and lithium; hydroxides of calcium and magnesium; hydroxides of other metals, such as aluminum and zinc; ammonia; and organic amines, such as unsubstituted or hydroxy substituted mono-, di-, or trialkylamines; dicyclohexylamine; tributylamine; pyridine; N-methyl-N-ethylamine; diethylamine; triethylamine; mono-, bis- or tris-(2-hydroxy-lower alkyl amines), such as mono-bis- or tris-(2-hydroxyethyl)amine, 2-hydroxy-tert-butylamine, or tris-(hydroxymethyl)methylamine, N,N-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like.

### EXAMPLES

Details of specific practical implementation of the present disclosure will be further described in the following examples. It should be understood that these examples are for illustrative purposes only and are not meant to limit the scope of the compositions and methods described herein.

### Example 1

In the following example, a concentrated gel base composition (Formulation A) was prepared in accordance with an embodiment of the present disclosure. The characteristics of the resulting composition were assessed.

The ingredients set out in Table 1 were added into a dispersing container, and the container was placed in a planetary mixer (Mazerustar KK-300S). The parameters for operating the mixer were set, including revolution, rotation and time variables. The person of skill will readily understand that the use of a planetary mixer illustrates a non-limiting example of obtaining the base composition from the list of ingredients, but that other devices or approaches may also be used. For example, in a variant preparation method, the ingredients set out in Table 1 are added into a container and heated at a temperature of 40° C. for 5 minutes instead of being placed in a planetary mixer. This variant method also includes a last step of 20-30 minutes de-aeration step. The resulting gel base composition is substantially identical to the one obtained with the preparation method that makes use of a planetary mixer.

The resulting gel base composition appeared as a clear, shiny viscous gel having a pale yellow to amber color. The resulting gel base composition had a specific gravity of 0.99 to 1.10, a pH between 4.4 and 5.7 and an initial viscosity of 250,000 to 500,000 cPs measured with a Brookfield RVDV-E, T-B, at 0.5 rpm, 60 seconds, and at 25° C.

Ingredients of the Formulation A are indicated in Table 1 below.

TABLE 1

| | | Percent |
|---|---|---|
| Ingredients | INCI | Range (%) |
| Mannitol | Mannitol | 1-8 |
| k-Carrageenan gum | Carrageenan gum | 0.1-1.5 |
| Xanthan gum | Xanthan gum | 0.02-0.5 |
| Ceratonia siliqua seed | Locust bean gum | 0.01-0.4 |
| polysaccharides | | |
| Pullulan | Pullulan NF-USP | 5-20 |
| Glycerin | Glycerin | 1-8 |
| Purified water | Purified water | 61.6-92.87 |
| Tween ™ 80 | Polysorbate 80 | 0.2-1 |

Other formulations using substitute ingredients are also possible to obtain a base composition having similar characteristics, for example where mannitol can be partially or wholly replaced with sorbitol, xylitol or a combination thereof, where either or both Carrageenan gum and Xanthan gum can be partially or wholly replaced with Guar gum, Tragacanth gum, modified cellulose gum or a combination thereof, where pullulan can be partially or wholly replaced with poloxamers, maltodextrins, Eudragit or a combination thereof; where Glycerin can be partially or wholly replaced with Propylene glycol, polyethylene glycols, or a combination thereof, where Tween 80 can be partially or wholly replaced with Tween 20, Poloxamer 407, or a combination thereof.

### Example 2

In the following example, the concentrated gel base composition prepared in Example 1 was processed into a film dosage form.

The concentrated gel base composition was spread with the appropriate consistency and aspect (no-bubbles) on an aluminum sheet into pre-defined strip length with sufficient spacing in between the strips for packaging purposes. The strips were then cured at a pre-defined temperature of from about 40° C. to 60° C., where the actual temperature selected for the curing step is mostly dependent on the active ingredient being compounded, i.e., a thermolabile active ingredient may require lower temperatures which may cause the curing step to last longer before obtaining the desired film, whereas conversely, a more thermal-resistant active ingredient may allow higher temperatures which allows a shorter curing phase. Typically, the curing step may thus last for a time period of at least 30 minutes to obtain strips with optimal flexibility. By increasing the curing time, this might affect the physical characteristics of the finished product.

The cured strips were then submitted to an additional air-drying step at room temperature for about 5-10 min under a laminar hood or on the bench (if relative humidity is below 45%) to allow the glass and the material to cool down at room temperature.

A second foil sheet of the same dimensions as the previous sheet was then applied on top of the strips and all four edges were sealed using a bag-sealer: following the sealing lines on top of the top foil sheet, the inventors proceeded by sealing the top and bottom foil sheets together using the bag-sealer. After the sealing was completed, the strip-containing pouches were cut following the aforementioned sealing lines located on the top foil sheet.

### Example 3

In the following example, the concentrated gel base composition prepared in Example 1 was diluted to prepare the following mucoadhesive oral dosage film forms or mucoadhesive gel forms:

TABLE 2
Coenzyme Q10 15 mg oral film (about 100 units)

| Ingredient | Quantity |
|---|---|
| Coenzyme Q10 | 1.65 g |
| Propylene glycol | 0.55 g |
| Formulation A | 3.712 g |

TABLE 3
Melatonin 7.5 mg in oral film (about 40 units)

| Ingredient | Quantity |
|---|---|
| Melatonin | 0.825 g |
| Propylene glycol | 0.55 g |
| Formulation A | 8.351 g |

TABLE 4
Haloperidol 1 mg in oral film (40 units)

| Ingredient | Quantity |
|---|---|
| Haloperidol | 0.11 g |
| Propylene glycol | 0.55 g |
| Formulation A | 12.37 g |

TABLE 5
Ketoprofen 12.5 mg in oral film (20 units)

| Ingredient | Quantity |
|---|---|
| Ketoprofen | 1.342 g |
| Propylene glycol | 0.55 g |
| Formulation A | 4.78 g |

TABLE 6
Estriol 0.05% in Gel (about 50 g)

| Ingredient | Quantity | |
|---|---|---|
| Estriol (Micronized), USP | 0.025 | g |
| Glycerin, USP | 1 | g |
| Formulation A | 15 | g |
| Medisca VersaPro ™ Gel base | 33.975 | g |

TABLE 7
Estriol 0.1%, Testosterone 0.1% in Gel (about 50 g)

| Ingredient | Quantity | |
|---|---|---|
| Estriol (Micronized), USP | 0.05 | g |
| Testosterone (Micronized), USP | 0.05 | g |
| Glycerin, USP | 1 | g |
| Formulation A | 15 | g |
| Medisca VersaPro Gel base | 33.9 | g |

TABLE 8
Lidocaine hydrochloride 5%, Prilocaine hydrochloride
5%, Tetracaine hydrochloride 2% in Gel (about 30 g)

| Ingredient | Quantity | |
|---|---|---|
| Lidocaine HCl, USP | 1.5 | g |
| Prilocaine HCl, USP | 1.5 | g |
| Tetracaine HCl, USP | 0.6 | g |
| Glycerin, USP | 3 | g |
| Formulation A | 23.4 | g |

TABLE 9
Nifedipine 0.3% in Gel (about 60 g)

| Ingredient | Quantity |
|---|---|
| Nifedipine, USP | 0.18 g |
| Formulation A | 59.82 g |

### Example 4

In the present example, the concentrated gel base composition prepared in Example 1 was compared to commercial mucoadhesive gel Mucolox™ (Professional Compounding Centers of America—PCCA, USA). Mucolox is a Colorless, semi-translucent viscous liquid having a pH of 5-6, soluble in water, specific gravity of 0.9-1.15 and viscosity of 4000-12,000 cps. Mucolox consists of Water, isomalt, Glycerin, Poloxamer 407, Tamrindus Indica Seed polysaccharide, Sodium Hyaluronate, Zea Mays (Corn) Starch, Simethicone, Carbomer, Sodium Benzoate, Potassium Sorbate, and Disodium EDTA.

The test used for comparing the concentrated gel base composition prepared in Example 1 and Mucolox is a test assessing mucin interaction through bioadhesive force (measured in mPa).

The test was repeated three times and the results indicate that both samples showed similar mucin interaction.

### Example 5

In the present example, the concentrated gel base composition prepared in Example 1 was compared to the commercial mucoadhesive gel (Mucolox™, Professional Compounding Centers of America, Inc.) to characterize the rheological metrics most relevant to the behavior of the samples and to identify the degree of variation in these rheological properties.

To this end the following tests were performed:
1) Shear Rate Sweep—to investigate the viscosity of the samples over a wide range of relevant shear rates, this was performed at approximate body temperature.
2) Oscillation Stress Sweep—to identify the presence and strength of soft solid structure when the samples are under low stress conditions, results from this test are thought to be relevant to handling and first touch properties.
3) Oscillation Frequency Sweep—to provide a viscoelastic fingerprinting method to gain a deeper understanding of the nature of interactions present in a formulation, notably an understanding of “relax-ability” and timescale-dependency that can shed light on storage, handling and performance attributes.
4) Tribology—To investigate the lubricating properties of the samples when forced to flow under a defined load at a range of sliding speeds.

### Equipment and Methods

Rheological analyses 1 and 3 as listed above were performed using a research rheometer (DHR2, TA Instruments) fitted with a 40 mm diameter 0.5° cone and plate measuring system. For the oscillation stress sweeps a 40 mm diameter crosshatched plate measuring system was utilized, testing gap set to 500 m. A solvent trap cover was employed for all rheological analyses to minimize atmospheric exposure of the samples at the exposed edges.

Tribology testing was performed using the same instrument fitted with a custom 3 balls on plate setup with a pliant lower substrate.

### Sample Preparation

Mucin solutions were made using DI water and porcine gastric mucin (II) purchased from Sigma Aldrich. Each solution was made to a concentration of 10%, the pH adjusted to 6.2 using 0.5M NaOH solution before being diluted with DI water to a final concentration of 6% before use.

3 g of the prepared mucin solution was mixed with an equal weight of the sample under test, giving a final mucin concentration of 3% (w/w).

For control samples, the mucin solutions and samples under investigation were diluted to 50% (w/w) of their initial concentration using DI water. To remove changes in pH as a possible influence the dilutions were also adjusted to pH 6.2.

All prepared samples were allowed to equilibrate overnight at 5° C. before any analysis was conducted.

1) Shear Rate Sweep

Viscosity/shear profiling entails subjecting a material to a range of shear conditions and observing its viscosity throughout. From the resulting “flow curve” viscosity at any relevant shear rates or stresses and the degree of non-Newtonian (typically shear thinning) behavior exhibited by a material can be identified and quantified.

Controlled rate viscosity profiles, where shear rate is swept, typically across mid to high shear rates, are good for obtaining a rapid viscosity profile to correlate to a range of handling conditions, particularly where a material is forced to flow at certain rates through the action of pumps, coating equipment or manually applied forces.

In the present experiment, following a 30 s equilibration time at 37° C. the samples were exposed to a 30 s pre-shear at a rate of 0.1 s⁻¹, this led immediately into a shear rate sweep, 0.1 s⁻¹ to 1000 s⁻¹, logarithmically scaled, 6 points per decade of shear rate, shear applied for 30 s at each rate with viscosity calculated over the final 5 seconds of each step.

Results are reported in Table 10 below.

TABLE 10

| | Viscosity (Pa · s) | | | Viscosity (Pa · s) | | |
|---|---|---|---|---|---|---|
| | at 1 s⁻¹ | | | at 1000 s⁻¹ | | |
| Sample | Run 1 | Run 2 | Mean | Run 1 | Run 2 | Mean |
| Mucolox | 2.79 | 2.62 | 2.71 | 0.328 | 0.320 | 0.324 |
| Formulation A | 6.81 | 7.63 | 7.22 | 0.246 | 0.245 | 0.245 |

2) Oscillation Frequency Sweep

The oscillatory frequency sweep entails applying small, sinusoidal (clockwise then counterclockwise) strains to a sample, sweeping the frequency of oscillation and monitoring the resulting stress response, from which viscoelastic information can be gained. The test is used to identify the relative proportions of viscous or elastic behavior across a range of deformation timescales.

Results from the oscillatory frequency sweeps are usually presented as viscoelasticity v timescale profiles of storage (G′) and loss modulus (G″) v frequency. Storage and loss modulus are measures of the respective abilities for the material to store energy through elastic deformation or dissipate energy through viscous flow during each oscillatory deformation cycle. In simple terms the test can establish the “dominant viscoelastic response” of a material.

In the present experiment, following a 60 s equilibration time at 25° C. the samples were exposed to oscillatory frequency sweeps, 100 rad/s to 0.1 rad/s, points spaced logarithmically, 4 points per decade of frequency, 0.1% oscillation strain. Results are reported in Table 11 below.

TABLE 11

| Complex Modulus Plateau (Pa) | | | *Yield Stress (Pa) | | | Phase Angle Plateau (°) | | |
|---|---|---|---|---|---|---|---|---|
| Run 1 | Run 2 | Mean | Run 1 | Run 2 | Mean | Run 1 | Run 2 | Mean |
| 10.4 | 10.3 | 10.4 | 5.5 | 5.9 | 5.7 | 60.1 | 57.9 | 59.0 |
| 25.35 | 29.42 | 27.39 | 18.78 | 30.30 | 24.54 | 29.6 | 23.7 | 26.6 |

*Yield stress was quantified by fitting an onset point model to the phase angle data; this entailed fitting straight lines tangential to the low stress plateau and the inflection during the yield, the point at which these two lines cross was taken as the yield stress.

3) Oscillation Stress Sweep

The oscillation stress sweep test provides a simple quantification of the rigidity and strength of soft solid structure present throughout a sample. The test entails the application of small, incrementing sinusoidal (i.e. clockwise then counterclockwise) shear stresses to the sample whilst monitoring its resulting deformation and/or flow. In the early stages of the test the stress is sufficiently low to preserve structure. The presence of this structure is revealed by dominant elastic deformation (rather than viscous flow) signified by a phase angle plateau at low values. Phase angle is a measure of the relative dominance of elastic or viscous response of the sample and ranges from 0° for an ideal elastic material (i.e. a perfect solid) to 900 for an ideal viscous material (a perfect liquid). At this stage the sample rigidity, the complex modulus, also remains at a plateau value. As the test progresses the incrementing applied stress eventually disrupts sample structure as the yielding process progresses. This is manifested as a loss of elastic response (phase angle rises) and an accompanying decrease in rigidity (complex modulus decreases).

The oscillation stress sweep may also be presented as storage modulus (G′) and loss modulus (G″) as a function of applied stress. Storage and loss modulus are measures of the respective abilities for the material to store energy through elastic deformation or dissipate energy through viscous flow during each oscillatory deformation cycle.

In the present experiment, following a 60 s equilibration time at 25° C. the samples were exposed to an oscillatory stress sweep ranging from 0.1 Pa to 1000 Pa, 10 points per decade, 1 Hz oscillation frequency. A step termination was set such that if the oscillation strain exceeded 1500% at any point then the test would end prematurely.

4) Tribology

Rheology studies the flow and deformation of films of materials separating surfaces in relative motion. Tribology, on the other hand, is the study of the friction, lubrication and wear of interacting surfaces—in other words, surfaces in close contact. The term bio-tribology relates specifically to the interaction of soft, biological surfaces.

Unlike rheology testing, where the sample under test is held in a defined gap between surfaces moving relative to each other, tribology testing entails bringing those surfaces into contact under a defined pressure and sliding one against the other, measuring the frictional drag over a range of sliding speeds. The surfaces and/or any applied lubricating liquid form the test sample. Tribology results are often displayed in the form of a Stribeck curve.

The Stribeck curve is typically composed of three regions. At low speeds the surfaces are in close contact with asperities (surface roughness features) interlocking. Under these conditions, lubrication is low, so friction is high. As sliding speed is increased the lubricant entrained between the upper and lower surfaces creates hydrodynamic lift, resulting in increasing separation of the surfaces and subsequent decreasing frictional drag. Lubrication at this stage is known as mixed boundary-hydrodynamic lubrication. Eventually, as sliding speed is increased, a complete separation of the surfaces ensues. Friction reaches a minimum at this stage and the final part of the Stribeck curve anatomy is reached: hydrodynamic lubrication. From the key features of the Stribeck curve we can derive metrics that clearly differentiate between materials of differing lubricating qualities.

For thick films, rheology dominates the handling and spreading characteristics, whereas for thin films, tribology takes over, influencing slipperiness and friction reduction. The spreading of, for example, a topical product onto the skin, involves the progressive decrease of its film thickness throughout the application process. In fact this time-dependent transition from a thicker to thinner film occurs in many cases where biological surfaces interact, also occurring, for example, during oral processing of foods and beverages. Rheological profiling can therefore illuminate the early stages of the material usage and tribology can inform us on the latter stages.

In the present experiment, a tribology assembly was employed that comprised a geometry of 3 glass spheres that slides against a pliant lower substrate, under a defined load of 1N, onto which the sample has been spread. The rotational angular velocity is ramped from 0.05 rad/s to 20 rad/s, 8 points per decade, each point maintained for 20 s with the coefficient of friction averaged over the final 15 s. The lower plate was made to hold 37° C. throughout the analysis.

5) Rheological Synergism

For the mucoadhesion study the supplied samples were analysed both individually and when mixed with prepared mucin solutions. Quantifying zero shear viscosity (ηo) was the ideal aim for each sample; it is thought that viscosity values at very low shear rates are most relevant to mucoadhesion behaviours. This viscosity was entered into the following two equations to give the “rheological synergism parameters: Δηo and Δηo/ηo+1.

Δηo=ηo(mix)−(ηo(sample)+ηo(mucin))

Δηo/ηo+1, where ηo=ηo(sample)+ηo(mucin)

Where Δηo is the difference between the actual viscosity values of the samples mixed with mucin and the theoretical values; the theoretical values are defined as the sum of the ηo values of the sample and the mucin when analyzed individually.

Δηo/ηo+1 describes the relative rheological synergism, this expresses the relative increase in ηo with regards to the sample and mucin alone. A relative rheological synergism parameter of 1 would indicate that there was no increase observed when the sample was mixed with mucin, meaning there was no observable interaction with the mucin solution.

Whereas a value greater than one indicates some interaction with mucin; a value of 2 for example would mean the measured viscosity of the sample mixed with mucin is double what was expected.

A value of less than one indicates either the interaction is negligible and that the value should be treated as if it were 1, or if the value is significantly less than 1 then this could indicate some negative interaction occurring.

In the present experiment, rheological synergism parameters for test samples and for test sample/mucin mixes—mean of two analyses were performed.

A zero shear plateau was the ideal aim and this was quantified for the Mucolox samples using a Carreau model fit, however, for the concentrated gel composition of Example 1, no plateau was observed in the shear rate range applied. Therefore, for the concentrated gel composition of Example 1, the values were taken at 0.1 s⁻¹ instead.

### Observations

All three of the rheology tests performed identified clear and repeatable differences between the samples.

The shear rate sweeps reveal differences in the shear dependence of the two samples; at low shear rates the concentrated gel composition of Example 1 has a far higher viscosity than Mucolox but at the higher shear rates this is reversed.

The oscillation stress sweeps clearly show that the concentrated gel composition of Example 1 is a stronger and more rigid sample (higher yield stress and complex modulus plateau) and also that the concentrated gel composition of Example 1 has a more elastic structure (lower phase angle plateau). It must be noted that although Mucolox seems to display some form of yielding behavior it has a phase angle greater than 45°, indicating more liquid-like behavior, even in the plateau at low stresses.

The oscillation frequency sweeps reveal that the concentrated gel composition of Example 1 behaves as an elastic solid over the full range of frequencies applied (storage modulus>loss modulus), tending towards a crossover at the highest frequencies. Mucolox by comparison behaves as a viscous fluid over this same range.

The tribology analysis identifies very little difference between the two samples in terms of their lubricating properties, giving similar coefficients of friction over the full range of sliding speeds applied.

The results of the investigation into the mucin interactions of the samples indicate that both samples show some mucin interaction, however, the concentrated gel composition of Example 1 has a greater degree of mucin interactivity than Mucolox. This is thought to indicate a greater degree of mucoadhesion.

### Example 6

This example reports a case study to assess the effectiveness of an embodiment of the mucoadhesive composition of the present disclosure which contains estriol in the treatment of urogenital atrophy.

Since the mucoadhesive composition of the present disclosure can be applied on different type of mucosa including the vaginal mucosa, and because the mucoadhesive composition of the present disclosure does not contain any ingredients that are considered irritants (propylene glycol, parabens, and the like); the present inventors predicted that use of this composition in the treatment of urogenital atrophy would have low probability of increasing existing vaginal infection or even causing it.

The case study consisted of Local estrogen treatment of vaginal atrophy in postmenopausal women. The primary goal of the treatment was to improve qualitative evaluation, to alleviate the symptoms, and to potentially reverse the atrophic changes from estrogen deprivation.

The duration of the treatment was of 12 weeks including screening visit gynecological evaluation at the beginning, at 4 weeks and at the end of the treatment. The study was a comparison between subjects that were administered with the following compounded preparation:
1) VersaPro™ Cream (Medisca, Canada)/Estriol
2) VersaPro Cream/mucoadhesive gel base/Estriol
3) Mucoadhesive gel base/Estriol

The investigator obtained consent from the patients for treatment and to data collect. The dosage of estriol was 0.25 mg and the number of patients was of 3 women.

At week 1 (baseline), week 4 and 12, a qualitative and objective assessment of the vaginal health of the patient (including pH evaluation) was performed.

The criteria for selection of the patient was the following:
1) Preferably 50-79 year old women
2) Amenorrhea for at least 12 months
3) History or a recurrent issue with moderate to severe urogenital atrophy symptoms.

The symptoms of urogenital atrophy included chronic and progressive inflammation of the vagina, dry, glazed-looking vaginal epithelium; a thinning cervix; a loss of labial fat pad; or a vagina that had lost elasticity, had shortened, had narrowed, had become less distensible, and that could be easily traumatized and irritated.

The exclusion criteria was HRT treatment 8 weeks prior the start of the study, sensitivity to estrogen compounded formula, abnormal vaginal bleeding, vaginal infection, and use of hypertensive drugs.

E3 a final metabolite of estrogen synthesis is a short-acting estrogen, since it has the shortest receptor occupancy and lowest receptor affinity of all estrogens. Thus, some estrogenic effects can be observed following single administration of E3, whereas ‘late effects’, which are based on a longer receptor retention time, are seen only with estradiol.

### Evaluation

The qualitative and objective approach to evaluate the vaginal health of each patient at the baseline, week 4 and week 12 included
1) Qualitative evaluation using vaginal symptoms score (VSS) and profile of female sexual function (PFSF)
2) Objective evaluation using vaginal health index (VHI), endometrial thickness and dried urine spot hormone testing to evaluate hormone patient's level at each time points.

### Results

The following tables summarize urine hormone testing results obtained from a 64-year-old patient that underwent the above described treatment.

The treatment formulation was compounded with the mucoadhesive gel base of example 1 to include Estriol at a final concentration of 0.25 mg of Estriol. For 12 weeks, the patients applied the preparation daily at bedtime for 2 weeks followed by twice a week thereafter.

Gynecological evaluation and dried urine spot hormone evaluation were monitored at the beginning of the study, at 4 weeks and at the end of the study (12 weeks) to evaluate hormone patient's level at each time points.

At week 1, the following patient reported no menstrual cycles, reported significant symptoms of androgen deficiency and significant fatigue in the afternoon/evening, but not in the morning.

TABLE 12
Results at Week 1

| | | | | Postmen- |
|---|---|---|---|---|
| | | Result | Luteal* | opausal |
| Test | | (ng/mg) | Range | Range |
| Progesterone Metabolites (Urine) | | | | |
| b-Pregnanediol | Below luteal range | 97.0 | 600-2000 | 60-200 |
| a-Pregnanediol | Below luteal range | 9.0 | 200-740 | 15-50 |
| Estrogens and Metabolites (Urine) | | | | |
| Estrone(E1) | Below luteal range | 2.2 | 12-26 | 3.0-7.0 |
| Estradiol(E2) | Below luteal range | 0.29 | 1.8-4.5 | 0.2-0.7 |
| Estriol(E3) | Below luteal range | 2.9 | 5-18 | 0.6-4.0 |
| 2-OH-El | Below luteal range | 1.06 | 5.1-13.1 | 0.3-2.0 |
| 4-OH-El | Within luteal range | 0.14 | 0-1.8 | 0-0.3 |
| 16-0H-E1 | Below luteal range | 0.15 | 0.7-2.6 | 0.2-0.6 |
| 2-Methoxy-E1 | Below luteal range | 0.61 | 2.5-6.5 | 0.3-1.4 |
| 2-OH-E2 | Low end of luteal | 0.09 | 0-1.2 | 0-0.3 |
| | range | | | |
| 4-OH-E2 | Below luteal range | 0.00 | 0.15-0.5 | 0-0.1 |
| 2-Methoxy-E2 | Below luteal range | 0.2 | 0.3-0.7 | 0-0.4 |
| Total Estrogen | Below range | 7.6 | 35-70 | 4.0-15 |
| Androgens and Metabolites (Urine) | | | | |
| DHEA-S | Low end of range | 31.0 | 20-750 | |
| Androsterone | Below range | 163.0 | 200-1650 | |
| Etiocholanolone | Within range | 629.0 | 200-1000 | |
| Testosterone | Below range | 1.9 | 2-3-14 | |
| 5a-DHT | Low end of range | 0.2 | 0-6.6 | |
| 5a- | Below range | 2.0 | 12-30 | |
| Androstanediol | | | | |
| 5b- | Low end of range | 28.6 | 20-75 | |
| Androstanediol | | | | |
| Epi-Testosterone | Below range | 0.8 | 2-3-14 | |

TABLE 13

| | Additional Normal Ranges | | |
|---|---|---|---|
| | Follicular | Ovulatory | Oral Pg (100 mg) |
| b-Pregnanediol | 100-300 | 100-300 | 2000-9000 |
| a-Pregnanediol | 25-100 | 25-100 | 580-3000 |
| Estrone (El) | 4.0-12.0 | 22-68 | N/A |
| Estradiol (E2) | 1.0-2.0 | 4.0-12.0 | N/A |

The patient's progesterone metabolites show that she is in the menopausal range, as expected. Postmenopausal progesterone is produced by the adrenal glands (not the ovaries), so therapies that improve progesterone in cycling women may not help in menopause. Progesterone supplementation may be appropriate for improving energy, sleep and mood in post-menopausal women, even when their levels are normal for a menopausal woman and may be appropriate if estrogen levels are higher than the postmenopausal levels. *the Luteal Range is the premenopausal range. When patients are taking oral progesterone this range for progesterone metabolites is not luteal and reflects the higher levels expected when patients take oral progesterone. The ranges in the table below may be used when samples are taken during the first few days (follicular) of the cycle, during ovulation (days 11-14) or when patients are on oral progesterone.

TABLE 14
Week 4

| | | | | Postmen- |
|---|---|---|---|---|
| | | Result | Luteal* | opausal |
| Test | | (ng/mg) | Range | Range |
| Progesterone Metabolites (Urine) | | | | |
| b-Pregnanediol | Below luteal range | 71.0 | 600-2000 | 60-200 |
| a-Pregnanediol | Below luteal range | 12.0 | 200-740 | 15-50 |
| Estrogens and Metabolites (Urine) | | | | |
| Estrone(E1) | Below luteal range | 2.2 | 12-26 | 3.0-7.0 |
| Estradiol(E2) | Below luteal range | 0.34 | 1.8-4.5 | 0.2-0.7 |
| Estriol(E3) | Within luteal range | 14.9 | 5-18 | 0.6-4.0 |
| 2-0H-E1 | Below luteal range | 1.05 | 5.1-13.1 | 0.3-2.0 |
| 4-0H-E1 | Within luteal range | 0.12 | 0-1.8 | 0-0.3 |
| 16-0H-E1 | Below luteal range | 0.21 | 0.7-2.6 | 0.2-0.6 |
| 2-Methoxy-El | Below luteal range | 0.37 | 2.5-6.5 | 03-1.4 |
| 2-OH-E2 | Low end of luteal | 0.09 | 0-1.2 | 0-0.3 |
| | range | | | |
| 4-OH-E2 | Below luteal range | 0.00 | 0.15-0.5 | 0-0.1 |
| 2-Methoxy-E2 | Within luteal range | 0.6 | 0.3-0.7 | 0-0.4 |
| Androgens and Metabolites (Urine) | | | | |
| DHEA-S | Below range | 13.0 | 20-750 | |
| Androsterone | Below range | 145.0 | 200-1650 | |
| Etiocholanolone | Within range | 565.0 | 200-1000 | |
| Testosterone | Low end of range | 2.7 | 2-3-14 | |
| 5a-DHT | Low end of range | 0.8 | 0-6.6 | |
| 5a- | Below range | 3.8 | 12-30 | |
| Androstanediol | | | | |
| 5b- | Within range | 40.9 | 20-75 | |
| Androstanediol | | | | |
| Epi-Testosterone | Below range | 0.5 | 2-3-14 | |

TABLE 15
Week 12

| | | | | Postmen- |
|---|---|---|---|---|
| | | Result | Luteal* | opausal |
| Test | | (ng/mg) | Range | Range |
| Progesterone Metabolites (Urine) | | | | |
| b-Pregnanediol | Below luteal range | 59.0 | 600-2000 | 60-200 |
| a-Pregnanediol | Below luteal range | 18.0 | 200-740 | 15-50 |
| Estrogens and Metabolites (Urine) | | | | |
| Estrone(E1) | Below luteal range | 2.2 | 12-26 | 3.0-7.0 |
| Estradiol(E2) | Below luteal range | 0.4 | 1.8-4.5 | 0.2-0.7 |
| Estriol(E3) | Within luteal range | 7.7 | 5-18 | 0.6-4.0 |
| 2-0H-E1 | Below luteal range | 0.77 | 5.1-13.1 | 0.3-2.0 |
| 4-0H-E1 | Within luteal range | 0.09 | 0-1.8 | 0-0.3 |
| 16-0H-E1 | Below luteal range | 0.15 | 0.7-2.6 | 0.2-0.6 |
| 2-Methoxy-E1 | Below luteal range | 0.54 | 2.5-6.5 | 0.3-1.4 |
| 2-OH-E2 | Low end of luteal | 0.07 | 0-1.2 | 0-0.3 |
| | range | | | |
| 4-OH-E2 | Below luteal range | 0.00 | 0.15-0.5 | 0-0.1 |
| 2-Methoxy-E2 | Low end of luteal | 0.3 | 0.3-0.7 | 0-0.4 |
| | range | | | |
| Total Estrogen | Below range | 12.2 | 35-70 | 4.0-15 |
| Androgens and Metabolites (Urine) | | | | |
| DHEA-S | Below range | 14.0 | 20-750 | |
| Androsterone | Below range | 142.0 | 200-1650 | |
| Etiocholanolone | Within range | 381.0 | 200-1000 | |
| Testosterone | Low end of range | 4.6 | 2-3-14 | |
| 5a-DHT | Low end of range | 0.7 | 0-6.6 | |
| 5a- | Below range | 4.8 | 12-30 | |
| Androstanediol | | | | |
| 5b- | Low end of range | 23.1 | 20-75 | |
| Androstanediol | | | | |
| Epi-Testosterone | Below range | 2.1 | 2-3-14 | |

The following tables summarize the quantitative assessment of vaginal health using the Vaginal Health Index, which is a system used to evaluate vaginal elasticity, fluid volume, pH, epithelial integrity, and moisture on a scale of 1 to 5. Table 16 reports the results from week 1, table 17 reports the results from week 4, and table 18 reports the result from week 12. The characteristics which are underlined and bold are those that were determined by the medical doctor proceeding with the assessment.

TABLE 16
Week 1

| | 1 | 2 | 3 | 4 | 5 - |
|---|---|---|---|---|---|
| Elasticity | None | Poor | Fair | Good [symbol] | Excellent, |
| Fluid volume | None | Scant amount, vault | Superficial, amount, | Moderate amount | Normal amount |
| (pooling of | | not entirely covered | vault entirely covered | of dryness (small | (fully saturates |
| secretions) | | | | areas of dryness | on cotton-tip |
| | | | | on cotton-tip | applicator) |
| | | | | applicator) | |
| pH | >=6.1 | 5.6-6.0 | 5.1-5.5 | 4.7-5.0 | <4.6 |
| Epithelial | Petechiae noted | Bleeds with | Bleeds with scraping | Not friable; | Normal |
| integrity | before contact | light contact | | thin epithelium | |
| Moisture | None, surface | None, surface not | Minimal | Moderate | Normal |
| (coating) | inflamed | | | | |

[symbol]

indicates data missing or illegible when filed

TABLE 17
Week 4

| | 1 | 2 | 3 | 4 | 5 |
|---|---|---|---|---|---|
| Elasticity | None | Poor | Fair | Good | Excellent |
| Fluid volume | None | Scant amount, vault | Superficial, amount, | Moderate amount | Normal amount |
| (pooling of | | not entirely covered | vault entirely covered | of dryness (small | (fully saturates |
| secretions) | | | | areas of dryness | on cotton-tip |
| | | | | on cotton-tip | applicator) |
| | | | | applicator) | |
| pH | >=6.1 | 5.6-6.0 | 5.1-5.5 | 4.7-5.0 | <4.6 |
| Epithelial | Petechiae noted | Bleeds with | Bleeds with scraping | Not friable; | Normal |
| integrity | before contact | light contact | | thin epithelium | |
| Moisture | None, surface | None, surface | Minimal | Moderate | Normal |
| (coating) | inflamed | not inflamed | | | |

TABLE 18
Week 12

| | 1 | 2 | 3 | 4 | 5 |
|---|---|---|---|---|---|
| Elasticity | None | Poor | Fair | Good | Excellent |
| Fluid volume | None | Scant amount, vault | Superficial, amount, | Moderate amount | Normal amount |
| (pooling of | | not entirely covered | vault entirely covered | of dryness (small | (fully saturates |
| secretions) | | | | areas of dryness | on cotton-tip |
| | | | | on cotton-tip | applicator) |
| | | | | applicator) | |
| pH | >=6.1 | 5.6-6.0 | 5.1-5.5 | 4.7-5.0 | <4.6 |
| Epithelial | Petechiae noted | Bleeds with | Bleeds with scraping | Not friable; | Normal |
| integrity | before contact | light contact | | thin epithelium | |
| Moisture | None, surface | None, surface | Minimal | Moderate | Normal |
| (coating) | inflamed | not inflamed | | | |

The results show that delivery of low dose estriol compounded in the mucoadhesive gel base described herein was able to considerably improve the clinical signs and symptoms as well as the quality of life of menopausal women suffering from vaginal atrophy.

### Example 7

In the present example, the concentrated gel base composition prepared in Example 1 (i.e., “test gel”) was compared to the commercial mucoadhesive gel Mucolox™ (i.e., “comparative gel”) to characterize the compressive/adhesive properties of the gel.

The compressive and adhesive properties of the test gel and the comparative gel were measured using a TA-XT2i HR texture analyzer (Stable Micro Systems, United Kingdom) in a “compressive test” and an “adhesive test”.

Each gel was scooped into a ˜39 mm diameter plastic cup until a depth of about 20 mm was reached. Testing was performed at ambient temperature and humidity and was accomplished using a ½″ diameter stainless steel probe with a 1″ radius of curvature. Both materials were deformed to a depth of 15 mm using a probe speed of 2 mm/s, held at the 15 mm depth for 1 second, and then removed at 2 mm/s. After waiting 15 seconds (and without cleaning the probe) a second deformation was performed; these two deformations are referred to as A and B, respectively, for a given trial. Five trials were performed for both materials.

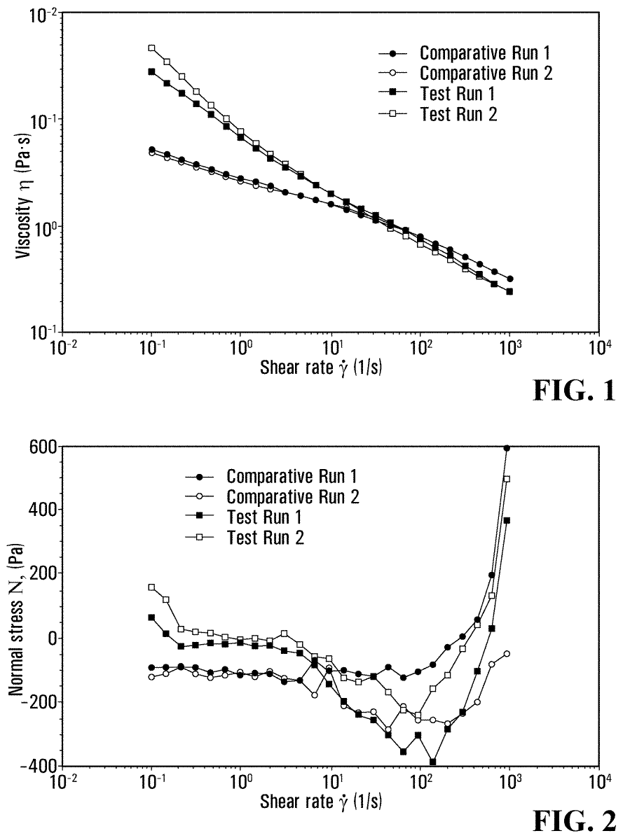

A typical load-time curve for the test gel is shown in FIG. 1. The scatter in the load curve is due to the relatively high data collection frequency used. From these data a maximum compressive force (MCF, observed during the indentation portion of deformation) and a maximum adhesive force (MAF, observed during the probe removal portion of deformation) can be determined. The MCF and MAF data for each deformation are summarized in Table 19. The initial and second deformations for a given trial were quite similar to each other. Average maximum compressive force (MCF) and maximum adhesive force (MAF) values of −9.0 and 3.0 g, respectively are observed. An overlay of the initial deformations from the five trails for the test gel is shown in FIG. 2. Excellent sample-to-sample reproducibility is observed.

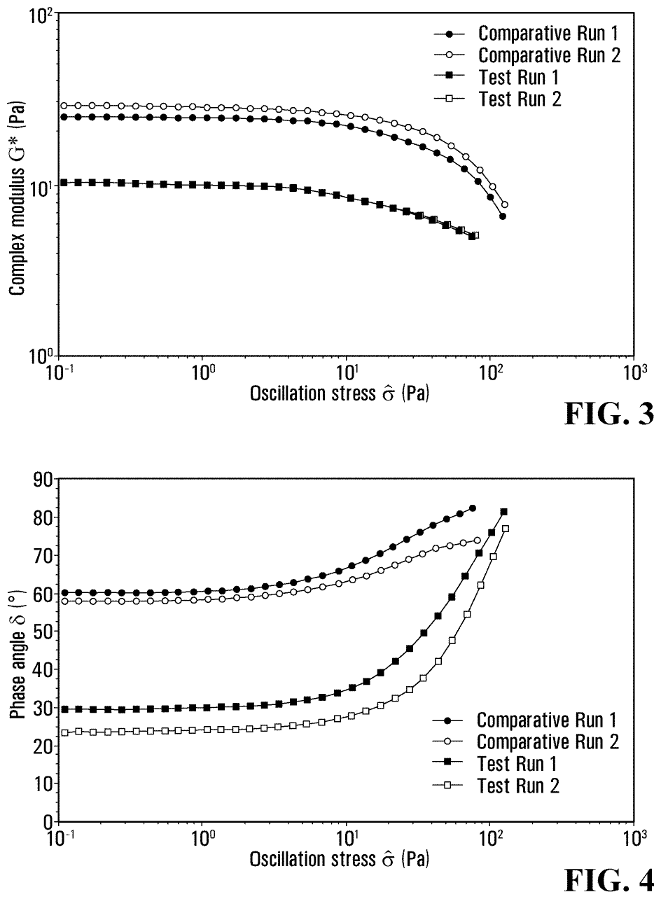

A typical load-time curve for the comparative gel is shown in FIG. 3. Again, scatter in the load data is due to the relatively high data collection frequency. Smaller MCF and MAF data are observed for this material relative to the test gel. Average MCF and MAF values of −2.3 and 1.5 g, respectively are observed. An overlay of the initial deformations from the five trails for the comparative gel material is shown in FIG. 4. Excellent sample-to-sample reproducibility is observed.

TABLE 19

| | | Max. Compressive | Max. Adhesive |
|---|---|---|---|
| Sample | Trial | Force [g] | Force [g] |
| Test gel | 1A | −8.4 | 3.5 |
| | 1B | −8.4 | 2.7 |
| | 2A | −8.2 | 3.0 |
| | 2B | −8.7 | 2.6 |
| | 3A | −9.5 | 3.3 |
| | 3B | −8.9 | 2.9 |
| | 4A | −9.1 | 3.5 |
| | 4B | −8.6 | 3.4 |
| | 5A | −10.2 | 3.0 |
| | 5B | −9.3 | 2.3 |
| | Average | −9.0 | 3.0 |
| | Std. Dev. | 0.6 | 0.4 |
| | Rel. Std. | 7 | 14 |
| | Dev. % | | |
| Comparative gel | 1A | −2.6 | 1.7 |
| | 1B | −2.0 | 1.4 |
| | 2A | −2.6 | 1.5 |
| | 2B | −1.3 | 1.5 |
| | 3A | −2.3 | 1.5 |
| | 3B | −1.9 | 1.4 |
| | 4A | −2.9 | 1.6 |
| | 4B | −1.2 | 1.2 |
| | 5A | −1.4 | 1.5 |
| | 5B | −4.4 | 1.8 |
| | Average | −2.3 | 1.5 |
| | Std. Dev. | 1.0 | 0.2 |
| | Rel. Std. | 42 | 11 |
| | Dev. % | | |

### Example 8

In the present example, the film dosage form prepared in Example 2 (i.e., “test strip”) was compared to the commercial Jamieson™ Melatonin Fast Dissolving Strips (Jamieson Laboratories Ltd., Canada) (i.e., “comparative strip”) to measure the tensile properties of the strips in a tensile test.

The tensile properties of both test strip and comparative strip were measured at ambient temperature and humidity on an RSA G2 rheometer (TA Instruments, USA) in a “tensile test”.

Prior to testing the strips were cut using a ½″ wide die. For both materials, the width and thickness of several specimens were measured and averaged. So as to minimize the time the strips were exposed to ambient conditions prior to testing, the average width and thickness measurements were used for all of the specimens. In this way the specimens could simply be loaded into the RSA G2 without spending time to measure their dimensions. The strips were kept in their sealed pouches until immediately prior to testing. A single pack of the comparative strips were used for the tensile testing and tested as quickly as possible to minimize exposure to ambient conditions. Data were collected using a 0.05 mm/s crosshead speed and a grip-to-grip separation of 1 cm. Seven (7) and nine (9) measurements were made for the test strip and comparative strip, respectively.

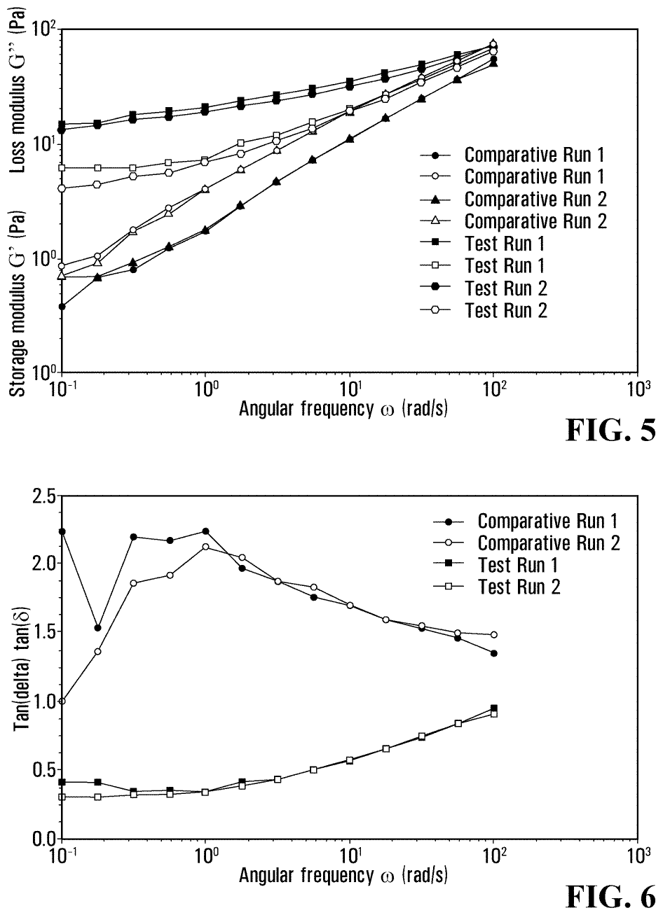

FIG. 5 is a typical load-deflection curve for a test strip specimen. The negative F(t) data in FIG. 5 are indicative of tensile forces. The maximum load (at break) and corresponding stress from each specimen is summarized in Table 20. An overlay of the load-time curves for the seven test strip specimens is shown in FIG. 6. The data are quite consistent from specimen-to-specimen. The breaks occurred near the grips. An average load at break of 1,273 g is observed, together with an average stress at break of 19.4×10⁷ dynes/cm².

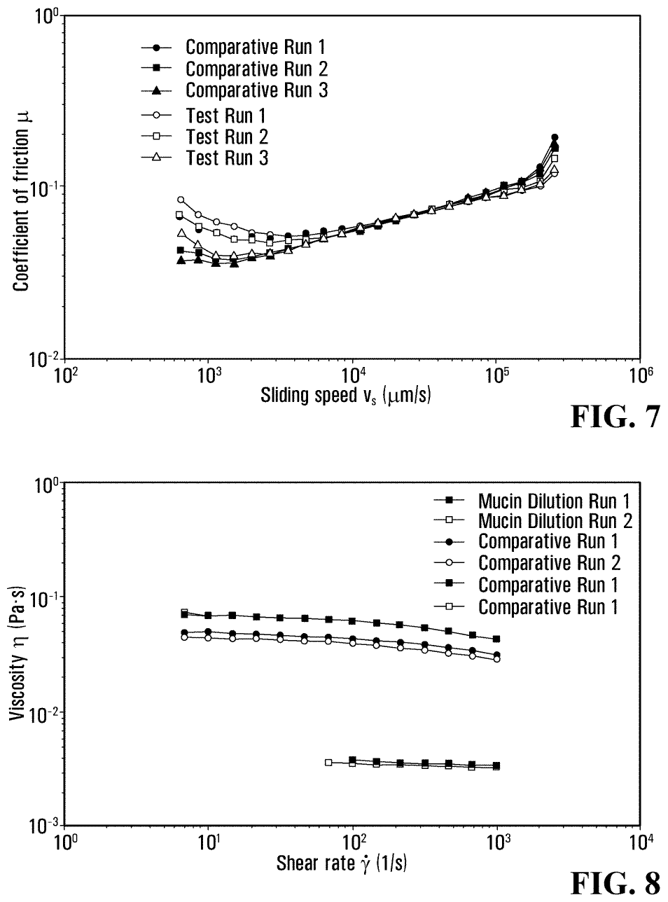

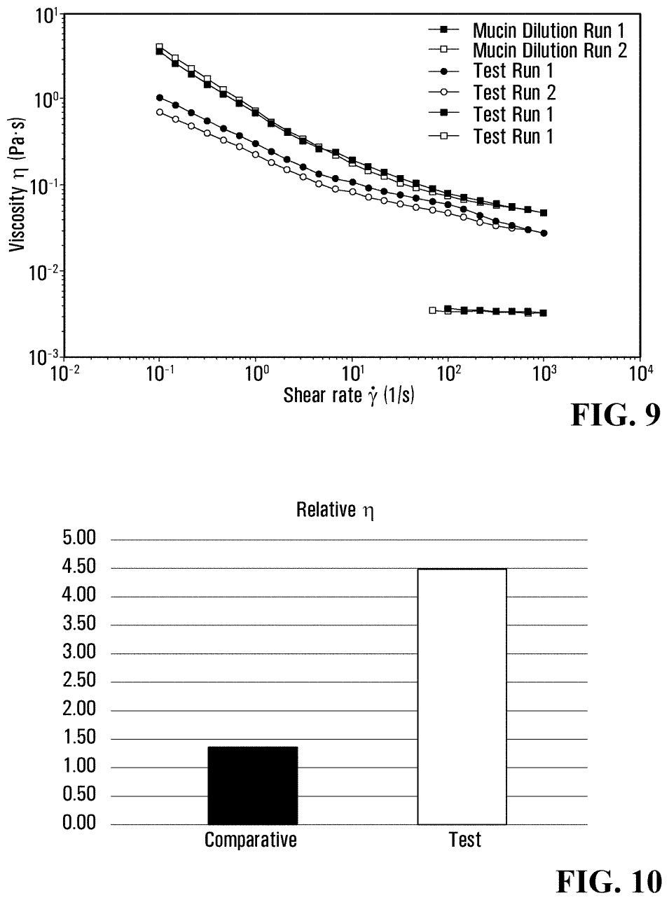

A typical load-deflection curve for a comparative strip is shown in FIG. 8. In contrast to the test strip, this load-deflection curve exhibits some curvature and is suggestive of some yield-type behavior. An overlay of the load-curves from all nine measurements of the comparative strips is shown in FIG. 9. The peak loads and stresses are also summarized in Table 20. There is more variability in these results compared to the test strips. Though the comparative strips used for the thickness measurements were consistent, it is possible that a larger sampling would have shown more variation. The breaks occurred near the grips. An average load at break of 895 g is observed, together with an average stress at break of 8.43×10⁷ dynes/cm².

TABLE 20

| | | Load at Break | Stress at break |
|---|---|---|---|
| Sample | Trial | [g] | [Dynes/cm²] |
| Test strip | 1 | 1,338 | 20.2 × 10⁷ |
| | 2 | 1,323 | 20.0 × 10⁷ |
| | 3 | 1,303 | 19.7 × 10⁷ |
| | 4 | 1,154 | 17.4 × 10⁷ |
| | 5 | 1,228 | 18.6 × 10⁷ |
| | 6 | 1,309 | 19.8 × 10⁷ |
| | 7 | 1,321 | 20.0 × 10⁷ |
| | Average | 1,273 | 19.4 × 10⁷ |
| | St. Dev. | 68 | 1.02 × 10⁷ |
| | Rel. Std. | 5.3 | 5.3 |
| | Dev. % | | |
| Comparative strip | 1 | 651 | 6.13 × 10⁷ |
| | 2 | 786 | 7.40 × 10⁷ |
| | 3 | 631 | 5.95 × 10⁷ |
| | 4 | 1,198 | 11.3 × 10⁷ |
| | 5 | 1,116 | 10.5 × 10⁷ |
| | 6 | 796 | 7.49 × 10⁷ |
| | 7 | 1,147 | 10.8 × 10⁷ |
| | 8 | 1,055 | 9.94 × 10⁷ |
| | 9 | 676 | 6.37 × 10⁷ |
| | Average | 895 | 8.43 × 10⁷ |
| | Std. Dev. | 231 | 2.18 × 10⁷ |
| | Rel. Std. | 26 | 26 |
| | Dev. % | | |

### Example 9

In the following example, the concentrated gel base composition prepared in Example 1 was used to prepare mucoadhesive oral dosage films by casting in prefabricated molds.

Square (3 cm×3 cm) and round (3 cm diameter) mold cavity samples or blister and proposed packaging components (for compounding Oral Film Strips) were sourced from Ideal Equipmentos para laboratorios, Brazil.

Preliminary oral film making trials were performed with undiluted concentrated gel base composition (100% gel) with or without API, in square or round molds with the following procedure:
1) Required quantity of undiluted or diluted concentrated gel base is weighed and loaded into a suitable mixing equipment. For example, a suitable mixing equipment includes any one of a mortar and pestle, a Samix mixer (Medisca Pharmaceutique Inc., Canada), a MAZ mixer (Medisca Pharmaceutique Inc., Canada), and the like. A suitable quantity of water-soluble color/dye may be added, if desired, for example for identification purposes.
2) The gel base is mixed at very low speed to a homogenous blend with minimum air bubble entrapment. If needed, air bubbles may be removed from the mixed composition by keeping at room temperature for about 1 to 2 hours in a closed container.
3) A suitable quantity (0.5 ml to 1 ml) of the homogenous blend is transferred into the cavity/well of the mold using a suitable manual or automatic dosing device. For example, a suitable dosing device may include a 1-ml Precise Dose syringe to transfer the homogenous blend. The gel is spread uniformly in the cavity/well of the mold with the syringe tip or by tilting the mold evenly on all directions. Alternatively, one can use any other mechanical/automatic device, such as a vibrating table.
4) The mold is placed and kept on a leveled base plate. The base plate can be a glass, metal, or plastic slab. To allow increased and more uniform hot air distribution, the base plate may include apertures to allow more uniform drying of the gel.
5) The loaded mold is transferred into a suitable drying device. For example, an air circulating oven, an ultrasound, a UV, an IR, or any other drying device. The drying device is operated for a sufficient time at a sufficient temperature to obtain the desired film. For example, a temperature of between about 40° C. and about 70° C. and a drying time period of between about 30 min to about 120 min.
6) The molds are taken out from the drying device and allowed to attain room temperature.
7) The dried films are peeled off from the mold. If desired, the dried films are kept inverted in the molds for about 5 to about 30 min to ensure that the bottom surface is sufficiently dried.
8) The dried films are wrapped in wax paper foil and packaged in sealed PE pouches or aluminum pouches. Alternatively, the whole mold containing the dried films, or the wrapped dry films can be packaged into a PE film holder, tray, or box, which is then packaged in the sealed pouch.

### Example 10

In this example, films were made according to the general procedure (with some minor modifications as required) set out in Example 9 and tested in the following preliminary trials to evaluate the feasibility of preparing medicated oral/topical films in a compounding pharmacy setting.

Table 21 reports results obtained from casting trials using the gel base composition without an API. The percentage of gel base composition conveys whether the gel base was diluted in water, e.g., a 75 wt. % gel base composition means that that 75 g. of gel base composition was diluted to 100 g. with water. The process parameters shown are drying temperature and drying time.

TABLE 21

| | Quantity in | | Mold | Process |
|---|---|---|---|---|
| Gel base | mold | | shape | parameters |
| 100% | 1 | ml | Square & | 40° C. & |
| | | | round | 150 min |
| 75% | 1 | ml | Square & | 40° C. & |
| | | | round | 200 min |
| 70% | 0.75 | ml | Square | 45° C. & |
| | | | | 75 min |
| 70% | 0.6 | ml | round | 45° C. & |
| | | | | 45 min |
| 70% | 0.6 | ml | square | 45° C. & |
| | | | | 50 min |
| 50% | 0.6 | ml | square | 45° C. & |
| | | | | 50 min |

Table 22 reports results obtained from casting trials using the gel base composition with an API (about 10 wt. % per film). The percentage of gel base composition conveys whether the gel base was diluted in water, e.g., a 75 wt. gel base composition means that 75 grams of gel base composition was diluted to 100 grams with water.

TABLE 22

| | | Quantity | | Mold | Process |
|---|---|---|---|---|---|
| Gel base | API | in mold | | shape | parameters |
| 50% | Caffeine | 0.6 | ml | square | 45° C. & |
| | | | | | 45 min |
| 50% | Caffeine | 0.6 | ml | square | 45° C. & |
| | | | | | 60 min |
| 50% | Caffeine | 0.6 | ml | square | 45° C. & |
| | | | | | 60 min |
| 50% | Clotrimazole | 0.6 | ml | square | 45° C. & |
| | | | | | 60 min |
| 100% | Caffeine | 0.6 | ml | square | 45° C. & |
| | | | | | 60 min |
| 50% | Phenylephrine HCl | 0.6 | ml | square | 45° C. & |
| | | | | | 60 min |
| 50% | Phenylephrine HCl | 0.6 | ml | square | 45° C. & |
| | | | | | 60 min |
| 50% | Phenylephrine HCl | 0.6 | ml | square | 45° C. & |
| | | | | | 75 min |
| 50% | Phenylephrine HCl | 0.6 | ml | square | 45° C. & |
| | | | | | 75 min |
| 70% | Phenylephrine HCl | 1 | ml | square | 45° C. & |
| | | | | | 60 min |
| 60% | Phenylephrine HCl | 1 | ml | square | 45° C. & |
| | | | | | 120 min |
| 60% | Diphenhydramine HCl | 1 | ml | square | 45° C. & |
| | | | | | 60 min |
| 60% | Cyclobenzaprine HCl | 1 | ml | square | 45° C. & |
| | | | | | 60 min |
| 70% | Cyclobenzaprine HCl | 1 | ml | square | 45° C. & |
| | | | | | 120 min |
| 70% | Diphenhydramine HCl | 1 | ml | square | 45° C. & |
| | | | | | 120 min |
| 70% + 10 mg | Cyclobenzaprine HCl | 1 | ml | square | 45° C. & |
| Flocel ™ 101 | | | | | 120 min |
| (MCC) | | | | | |
| 70% + 10 mg | Diphenhydramine HCl | 1 | ml | square | 45° C. & |
| Flocel ™ 101 | | | | | 120 min |
| (MCC) | | | | | |

### Example 11

In this example, the base gel composition of Example 1 and comparative gel Mucolox (Professional Compounding Centers of America, Inc.) were processed with the film dosage form manufacturing process set out in Example 9.

The general film casting procedure set out in Example 9 was followed using 1 ml each of the diluted (60%) base gel composition of Example 1 (“test strip”) and 100% Mucolox (“comparative strip”) with a square mold and with the following drying parameters: 45° C. for 60 minutes or 120 minutes.

The average moisture content of the test strip was 118% and 104% at 1 hour and 2 hours drying, respectively. The theoretical moisture content of the comparative strip could not be calculated as the total solid content is not available. However, the total dry weight of the comparative strip was about 3.3 times more than the test strip for both drying times. Moreover, the comparative strips were sticky and not easily peelable from the mold and crumbled when taken out of the mold whereas the test strip were easily peelable.

### Example 12

In the present example, the base gel composition of Example 1 was processed with the film dosage form manufacturing process set out in Example 9. Different drying time were tested.

Table 23 reports results obtained from casting trials with variable drying time using the gel base composition without an API. The percentage of gel base composition conveys whether the gel base was diluted in water, e.g., a 75 wt. % gel base composition means that that 75 g. of gel base composition was diluted to 100 g. with water. The process parameters shown are drying temperature and drying time.

TABLE 23

| | Quantity | Mold | Process | Calculated |
|---|---|---|---|---|
| Gel base | in mold | shape | parameters | Weight |
| 100% | 1 mL | Square | 45° C. | 1 h - 125% |
| | | | 1, 2, 3, or 4 h | 2 h - 114% |
| | | | | 3 h - 111% |
| | | | | 4 h - 110% |
| 70% | 1 mL | Square | 45° C. | 1 h - 118% |
| | | | 1, 2, 3, or 4 h | 2 h - 110% |
| | | | | 3 h - 108% |
| | | | | 4 h - 107% |
| 60% | 1 mL | Square | 45° C. | 1 h - 113.5% |
| | | | 1, 2, 3, or 4 h | 2 h - 108% |
| | | | | 3 h - 106.5% |
| | | | | 4 h - 106.5% |
| 50% | 1 mL | Square | 45° C. | 1 h - 115.5% |
| | | | 1, 2, 3, or 4 h | 2 h - 108% |
| | | | | 3 h - 108% |
| | | | | 4 h - 107% |

The film drying rate studies indicate that most of the drying occurs in the first 2 hours of drying and no significant loss of moisture beyond 2 hours of drying at 45° C. The best film in terms of appearance at drying for 1 h to 2 h seems to be 60%-50% gel base with quantity of 1 ml. Lower than 60% gel base showed no significant difference in the drying rate.

Actual dry weights of the films varied from about 105% to 125% of the theoretical weight depending upon the drying temperature and time. Most of the films for any dilution or concentration were showing about 115% of the theoretical weight for 1 hour drying at 45° C.

### Example 13

In the present example, the base gel composition of Example 1 was processed with the film dosage form manufacturing process set out in Example 9 using 10 unit/cavity trays prefabricated molds.

The 10 unit/cavity mold tray samples are thicker and the mold cavity has less depth (about 0.7 mm), compared to the cavity depth of a single unit mold (about 1.5 mm) and, therefore, only 0.6 to 0.8 ml of solution would fill into each mold cavity of the 10 unit tray without overfilling. However, for this trial purpose the mold cavities were carefully filled and handled with 1 ml gel along with the lower fill volume trials (0.8 and 0.6 ml). The percentage theoretical weight value is the percentage actual dry weight of the film obtained compared to theoretical dry weight.

TABLE 24

| Actual Dry | % | Actual Dry | % | Actual Dry | % | Actual Dry | % |
|---|---|---|---|---|---|---|---|
| weight of | theor. | weight of | theoretical | weight of | theoretical | weight of | theoretical |
| the film | weight | the film | weight | the film | weight | the film | weight |
| 100% gel - 1 ml/mold - | | 80% gel - 1 ml/mold - | | 70% gel - 1 ml/mold - | | 60% gel - 1 ml/mold - | |
| 100 min drying @45° C. | | 60 min drying @45° C. | | 60 min drying @45° C. | | 60 min drying @45° C. | |
| 0.2086 | 117.32 | 0.1660 | 116.70 | 0.1425 | 114.49 | 0.1251 | 117.27 |
| 0.2074 | 116.65 | | | 0.1418 | 113.93 | 0.1225 | 114.83 |
| 0.2125 | 119.52 | | | **0.14215 | **114.21 | **0.1238 | **116.05 |
| **0.2095 | **117.83 | | | | | | |
| 100% v - 0.8 ml/mold - | | 80% gel - 0.8 ml/mold - | | 70% gel - 0.8 ml/mold - | | 60% gel - 0.8 ml/mold - | |
| 100 min drying @45° C. | | 60 min drying @45° C. | | 60 min drying @45° C. | | 60 min drying @45° C. | |
| 0.1617 | 113.68 | 0.1315 | 115.56 | 0.1119 | 112.39 | 0.0954 | 111.78 |
| 0.1623 | 114.10 | | | 0.1139 | 114.39 | 0.0947 | 110.96 |
| 0.1626 | 114.31 | | | 0.1120 | 112.49 | 0.0960 | 112.49 |
| **0.1622 | **114.03 | | | **0.1126 | **113.09 | **0.095367 | **111.74 |
| 100% gel - 0.6 ml/mold - | | 80% gel - 0.6 ml/mold - | | 70% gel - 0.6 ml/mold - | | 60% gel - 0.6 ml/mold - | |
| 60 min drying @45° C. | | 60 min drying @45° C. | | 60 min drying @45° C. | | 60 min drying @45° C. | |
| 0.1197 | 112.20 | 0.0997 | 116.82 | 0.0853 | 114.23 | 0.0737 | 115.14 |
| 0.1229 | 115.20 | | | 0.0863 | 115.57 | 0.0734 | 114.67 |
| 0.1237 | 115.95 | | | 0.0845 | 113.16 | 0.0733 | 114.52 |
| **0.1221 | **114.45 | | | **0.085367 | **114.32 | **0.073467 | **114.78 |

**Average of the actual dry weight of the films and the average of the theoretical dry weights based on the volume of the gel added into the mold cavity.

The above data indicates that 100% gel base in the 10-unit tray mold requires more time compared to the previous trials with single unit mold. Also, the thickness of the 10-unit tray mold cavity appears to be more than of the thickness of the single unit mold. This may be one of the reasons for slower drying rate in the 10-unit tray mold.

### Example 14

In the present example, the base gel composition of Example 1 was processed with the film dosage form manufacturing process set out in Example 9 and the water activity was measured.

A preliminary assessment of water activity of films was done using different gel base concentrations (100%, 80%, 70% and 60%) and different volumes of (1 ml, 0.8 ml and 0.6 ml) in a bracketed testing design.

All the samples were uniformly dried at 45° C. for 60 minutes in an oven in single unit molds on a 1″ glass base plate. 25 films of each concentration and the corresponding dilution were prepared and kept at room temperature for about 2 hours after taking out from the drying chamber. The dried films were wrapped in wax paper foil, packaged in polyethylene pouches and identified with a label. After 2 days, 20 of the wrapped films of each sample were packaged in an aluminum pouch, heat-sealed, labelled and sent for water activity testing.

The results from two assays are reported in Table 25.

TABLE 25

| | | Average dry weight of | |
|---|---|---|---|
| | | the film & % theoretical | |
| | | dry weight of the | |
| Sample description | | film (25 films) | Water activity |
| 100%/1 | ml | 0.2055 g = 115.57% | 0.598 and 0.599 |
| 100%/0.6 | ml | 0.1216 g = 113.98% | 0.488 and 0.488 |
| 80%/0.8 | ml | 0.1334 g = 117.21% | 0.546 and 0.546 |
| 60%/1 | ml | 0.1231 g = 115.4% | 0.533 and 0.532 |
| 60%/0.6 | ml | .07437 g = 116.2% | 0.500 and 0.502 |

The a_{w} results indicates that the water activity of the films are close to the limiting value of ≤0.6, which means that the films are good for microbial stability.

Other examples of implementations will become apparent to the reader in view of the teachings of the present description and as such, will not be further described here. Note that titles or subtitles may be used throughout the present disclosure for convenience of a reader, but in no way, these should limit the scope of the invention. Moreover, certain theories may be proposed and disclosed herein; however, in no way they, whether they are right or wrong, should limit the scope of the invention so long as the invention is practiced according to the present disclosure without regard for any particular theory or scheme of action. All references cited throughout the specification are hereby incorporated by reference in their entirety for all purposes.

It will be understood by those of skill in the art that throughout the present specification, the term “a” used before a term encompasses embodiments containing one or more to what the term refers. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this invention pertains. In the case of conflict, the present document, including definitions will control.

As used in the present disclosure, the terms “around”, “about” or “approximately” shall generally mean within the error margin generally accepted in the art. Hence, numerical quantities given herein generally include such error margin such that the terms “around”, “about” or “approximately” can be inferred if not expressly stated.

Although various embodiments of the disclosure have been described and illustrated, it will be apparent to those skilled in the art in light of the present description that numerous modifications and variations can be made. The scope of the invention is defined more particularly in the appended claims.

1. A composition for use in formulating a mucoadhesive delivery dosage form, the composition having a viscosity of at least 50,000 cPs, wherein
upon spreading the composition as a layer on a substrate, the composition optionally having been diluted with a diluting agent, and drying the composition, the composition being compoundable into a mucoadhesive film strip dosage form, and
upon dilution with a diluting agent, the composition being compoundable into a mucoadhesive gel dosage form.

2. The composition according to claim 1, comprising an active ingredient.

3. The composition according to claim 2, wherein the active ingredient includes an active pharmaceutical ingredient (API), a nutraceutical compound, a cannabinoid, a cosmetic compound, or a combination thereof.

4. The composition according to claim 3, the film strip dosage form having a water activity of ≤0.6.

5. The composition according to claim 4, the film strip dosage form having a water activity of ≥0.04.

6. The composition according to claim 1, the composition having a viscosity of at least 80,000 cPs.

7. The composition according to claim 6, the viscosity being of at least 100,000 cPs.

8. The composition according to claim 6, the viscosity being of at least 200,000 cPs.

9. The composition according to claim 6, the viscosity being of at least 300,000 cPs.

10. The composition according to claim 1, the composition having an initial viscosity and the gel dosage form having a target viscosity which is reduced by at least 20% compared to the initial viscosity.

11. The composition according to claim 1, wherein the composition comprises a mucoadhesive polymer.

12. The composition according to claim 11, wherein the mucoadhesive polymer includes a natural, semisynthetic or synthetic polymer.

13. The composition according to claim 12, wherein the mucoadhesive polymer includes amylopectin, zein, modified zein, casein, gelatin, serum albumin, collagen, chitosan, pyrrolidones, dextrins, cellulose, dextrans, tamarind seed polysaccharide, gellan, carrageenan gum, xanthan gum, arabic gum, hyaluronic acid, polyhyaluronic acid, alginic acid, locust bean gum, pullulan, poloxamers, maltodextrins, Eudragit, guar gum, tragacanth gum, modified cellulose gum, or any combinations thereof.

14. The composition according to claim 12, wherein the mucoadhesive polymer includes carrageenan gum, xanthan gum, locust bean gum, pullulan, and any combinations thereof.

15. The composition according to claim 14, wherein the composition further comprises a plasticizer.

16. The composition according to claim 15, wherein the plasticizer includes glycerin, alkylene glycols, polyalkylene glycols, glycerol, triacetin, deacetylated monoglyceride, diethyl salate, triethyl citrate, dibutyl sebacate, polyethylene glycols, propylene glycol, or any combinations thereof.

17. The composition according to claim 15, wherein the plasticizer is glycerin.

18. The composition according to claim 17, wherein the composition further comprises a pharmaceutically acceptable polyhydric alcohol.

19. The composition according to claim 18, wherein the pharmaceutically acceptable polyhydric alcohol includes mannitol, glucose, sucrose, dextrose, sorbitol, xylitol, maltitol, erythritol, or any combinations thereof.

20. The composition according to claim 18, wherein the pharmaceutically acceptable polyhydric alcohol is mannitol.

21. The composition according to claim 11, wherein the composition further comprises an emulsifier.

22. The composition according to claim 21, wherein the emulsifier includes a poloxamer, benzalkonium chloride, polysorbate, sodium lauryl sulfate, or any combinations thereof.

23. The composition according to claim 21, wherein the emulsifier is polysorbate 80.

24. The composition according to claim 1, wherein the diluting agent includes a carrier, excipient, or diluent.

25. The composition according to claim 24, wherein the diluting agent includes water.

26. The composition according to claim 1, wherein the diluting agent is a liquid, cream or gel.

27. A composition for use in formulating a mucoadhesive delivery dosage form, the composition having a viscosity of at least 50,000 cPs, wherein
upon dilution with a diluting agent, the composition is in a gel dosage form, and
upon spreading the composition as a layer on a substrate, the composition optionally having been diluted with a diluting agent, and drying the composition, the composition is in a film strip dosage form.

28. The composition according to claim 27, comprising an active ingredient.

29. The composition according to claim 28, wherein the active ingredient includes an active pharmaceutical ingredient (API), a nutraceutical compound, a cannabinoid, a cosmetic compound, or a combination thereof.

30. The composition according to claim 29, the film strip dosage form having a water activity of ≤0.6.

31-219. (canceled)
